(19) United States
(12) Patent Application Publication
(10) Pub. No.: US 2023/0088285 A1
(43) Pub. Date: Mar. 23, 2023

(54) ABDOMINAL AORTIC ANEURYSM GROWTH PROGRESSION

(71) Applicant: Oxford University Innovation Limited, Oxford (Oxfordshire) (GB)

(72) Inventor: Regent LEE, Oxford (Oxfordshire) (GB)

(21) Appl. No.: 17/800,562

(22) PCT Filed: Mar. 1, 2021
(86) PCT No.: PCT/GB2021/050511

§ 371 Date: Aug. 18, 2022

(30) Foreign Application Priority Data
Feb. 28, 2020 (GB) 2002930.2
Sep. 14, 2020 (GB) 2014375.6

(51) Int. Cl. G01N 33/68

(52) U.S. Cl. CPC G01N 33/68; G01N 2800/329; G01N 2800/50

(57) ABSTRACT

The present invention relates to aortic aneurysm growth progression and predicting future progression of abdominal aortic aneurysms. The present invention concerns the use of at least one protein selected from at least one of four groups of proteins as a biomarker for determining a risk value of abdominal aortic aneurysm future growth for a subject. The groups are: a group of proteins determined to be present at higher concentrations in subjects showing fast abdominal aortic aneurysm growth compared with subjects showing slow abdominal aortic aneurysm growth; a group of proteins determined to be significantly lower in the systemic circulation of subjects following abdominal aortic aneurysm surgery; a group of proteins determined to be present in thrombus of an abdominal aortic aneurysm; and a group of proteins determined to be present in supernatant of an extracted thrombus sample.

The present invention relates to aortic aneurysm growth progression and to materials, apparatus and methods for determining a risk factor or value indicative of predicted growth of an abdominal aortic aneurysm of a patient. In particular, the aspects of the invention may be used to predict future progression of abdominal aortic aneurysms.

### BACKGROUND

Abdominal aortic aneurysms (AAA) are pathological dilatations of the abdominal aorta which can result in rupture and mortality. Patients with AAAs have an increased risk of cardiovascular morbidity. Aortic aneurysms, in particular abdominal aortic aneurysms, are associated with biological changes in the vasculature, features of systemic inflammation and endothelial dysfunction. Rupture of an AAA results in death, even in >50% of those who receive prompt surgery. AAAs may present in a subject in a variety of sizes. An AAA is typically defined as a region of an abdominal aortic artery having an outer aortic diameter greater than 30 millimetres. If the outer diameter of the aorta in the region of the aneurysm is 55 millimetres or greater, the abdominal aortic aneurysm is considered to be large. Surgery to treat AAAs is considered where an AAA is identified having a diameter of 55 millimetres or greater because the risk of rupture of the AAA is greater than the traditional risks associated with surgery. In current practice, surgery is typically not considered where the identified AAA has a diameter less than 55 millimetres as the risks of surgery are generally considered to outweigh the risk of aneurysm rupture. Currently, where an AAA is identified in a patient, and the AAA has a size between approximately 30 millimetres and 55 millimetres, the size of the AAA is monitored over time. Aneurysms may grow at different rates, if at all. If the aneurysm grows to exceed 55 millimetres, then surgery will typically be performed to treat the aneurysm.

In our earlier application, WO 2017/212210, to which further reference should be made, we observed accelerated systemic endothelial dysfunction as measured by brachial artery flow-mediation vasodilation (FMD) in AAA patients and were able to correlate FMD values with future AAA growth.

### BRIEF SUMMARY OF THE DISCLOSURE

AAAs have been shown to contain intra-luminal thrombus (ILT). It can also be observed that systemic endothelial dysfunction is reversed by AAA repair. Since ILT is either removed or excluded from circulation after successful repair of AAAs, the present inventors have hypothesised ILT to be the source of mediators that contribute to AAA growth. It is with this in mind that the present invention has been devised.

In one aspect, the present invention provides the use of at least one protein selected from at least one of Group A, Group B, Group C and/or Group D as a biomarker for determining a risk value of aneurysm future growth for a subject.

Group A is a group of proteins determined to be present at differentially expressed concentrations in subjects showing fast aneurysm growth compared with subjects showing slow aneurysm growth.

Group B is a group of proteins determined to be significantly different in the systemic circulation of subjects following aneurysm surgery.

Group C is a group of proteins determined to be present in thrombus of an aneurysm.

Group D is a group of proteins determined to be present in supernatant of an extracted thrombus sample (in other words, is released from the thrombus).

In another aspect, the present invention provides a method of determining a risk value of future aneurysm growth for a subject, the method comprising receiving a blood sample of the subject, determining a protein concentration in the blood sample for at least one protein selected from at least one of Group A, Group B, Group C and/or Group D as defined in claim 1; comparing the determined protein concentration with a reference value for the protein and an index of aneurysm growth for the protein; and determining the risk value of future aneurysm growth based on the comparison.

In a further aspect, the present invention provides an apparatus for determining a risk value of future aneurysm growth for a subject, the apparatus comprising a data input to receive at least one value of protein concentration of a blood sample of the subject, the protein being at least one protein selected from at least one of Group A, Group B, Group C and/or Group D as defined in claim 1; at least one processor; and a memory comprising instructions executable by the at least one processor to: i) compare the or each protein concentration with a respective protein reference value and an index of aneurysm growth for the protein; and ii) determining the risk value of future aneurysm growth based on the comparison.

Optionally, the apparatus further comprises a blood sample analysis module.

Preferably, the at least one protein is selected from at least two of Group A, Group B, Group C and Group D; or at least three of Group A, Group B, Group C and Group D; or is selected from Group A, Group B, Group C and Group D.

In certain examples, the at least one protein is at least one protein selected from proteins present in both Group A and Group B.

In certain examples, the at least one protein is at least one protein selected from proteins present in both Group A and Group B and at least one of Group C and Group D.

In a preferred embodiment, the at least one protein is at least one protein selected from proteins present in Group A, Group B, Group C and Group D.

Preferably, the at least one protein is at least one of attractin, Apolipoprotein A4, Complement C8 and HSP90AA5P.

In certain examples, Group A is a group of proteins as set out in Table 1 below; and/or Group B is a group of proteins as set out in Table 2 below; and/or Group C is a group of proteins as set out in Table 3 below; and/or Group D is a group of proteins as set out in Table 4 below.

### BRIEF DESCRIPTION OF THE DRAWINGS

The above and other aspects of the present invention will now be described in further detail, by way of example only, with reference to the following examples and the accompanying drawings, in which:

FIG. 1 is a Venn diagram illustrating protein groups A, B, C and D of the present invention and their overlap;

FIG. 2 shows the relationship between blood attractin level and AAA progression;

FIG. 3 shows AAA growth plotted against blood attractin level;

FIG. 4 shows the results of using attractin level and AAA diameter as input variables in the methods of the present invention, showing the AUROC for predicting NO growth of AAA at 12 months is 85%;

FIG. 5 shows the results of using attractin level and AAA diameter as input variables in the methods of the present invention, showing the AUROC for predicting FAST growth of AAA at 12 months is 76%;

FIG. 6 shows the results using i) attractin level, ii) AAA diameter, and iii) attractin level+AAA diameter in combination, as input variables in the methods of the present invention, showing the AUROC for predicting fast growth of AAA at 12 months is 76% (Attractin level alone), 52% (AAA diameter), and 76% (Attractin level+AAA diameter);

FIG. 7 shows the results using i) attractin level, ii) AAA diameter, and iii) attractin level+AAA diameter in combination, as input variables in the methods of the present invention, showing the AUROC for predicting slow growth of AAA at 12 months is 69% (Attractin level alone), 76% (AAA diameter), and 85% (Attractin level+AAA diameter);

FIG. 8 shows AAA growth plotted against blood hepcidin level;

FIG. 9 is a diagram showing an apparatus in accordance with an example of the present invention; and

FIG. 10 is a flow diagram illustrating a method in accordance with an example of the present invention.

### DETAILED DESCRIPTION

In accordance with the present invention there is provided a method for determining a risk value indicative of predicted growth of an abdominal aortic aneurysm of a patient. The method comprises determining or receiving a value representative of or representing a blood concentration of at least one protein which the present inventors have determined to be indicative biomarkers of aneurysm progression. The method also comprises determining a risk value indicative of predicted growth by evaluating the received values with reference values in an aneurysm risk model.

The aneurysm risk model relates to a risk value indicative of predicted growth of an abdominal aortic aneurysm for a given value representative of or representing the size of an abdominal aorta/aortic aneurysm and a given protein concentration. The value representative of or representing the size of an abdominal aortic aneurysm may be the size of the aneurysm.

The reference value may be an average or expected value for the index of blood protein concentration.

In some embodiments, the aneurysm risk model is a look-up table.

The risk value indicative of predicted growth of an abdominal aortic aneurysm of a patient may be a qualitative (e.g. fast, medium or slow) or quantitative (e.g. mm per unit time or percentage per unit time) measurement.

In current practice, the size of a patient's abdominal aortic aneurysm is used to guide a decision on whether or not the abdominal aortic aneurysm should be surgically repaired. As mentioned above, surgical intervention is recommended once the aneurysm reaches a surgical threshold size of, for example, 55 mm. Below this threshold, the risks of surgery are generally considered to outweigh the benefits of aneurysm removal.

It has been found that this prior approach has intrinsic shortcomings because aneurysm size may not an absolute predictor of the risk of rupture. Furthermore, the rate of AAA progression may vary significantly between individuals. For example, there may be a risk of rupture in those individuals with relatively smaller AAAs (e.g. 35-55 mm or 40-55 mm), and many patients with an initially small/moderate size AAA may progress and require surgery within 5 years. This may be an important consideration in ageing populations, as the risks associated with undergoing interventions increase with age.

Thus, in a preferred embodiment, the method is used to determine a risk value indicative of predicted growth of an abdominal aortic aneurysm of a patient identified as having an abdominal aorta/aortic aneurysm that is less than 55 mm, for example, 30 to 55 mm, preferably 35 to 55 mm, more preferably 40 to 55 mm in size. Thus, the method may comprise receiving a value representative of or representing a size of the abdominal aorta/aortic aneurysm, wherein the size of the abdominal aorta is less than 55 mm, for example, 30 to 55 mm or, preferably, 35 to 55 mm or 40 to 55 mm. The subgroup of patients with AAA size between 35 and 55 mm or preferably 40 and 55 mm may be particularly important in terms of potential for change in clinical practice. Surgeons may be convinced to provide surgical intervention in this subgroup (e.g. 35-40 mm or 40-55 mm) of patients when there is sufficient justification based, for example, on predicted growth rate.

The method of the present invention may further comprise guiding a decision on a clinical intervention of the patient based on the determined risk value. For example, if the determined risk value of a patient is high, the patient's aneurysm may be fast-growing. Thus, the patient in question may benefit from surgery even though his/her aneurysm is below a surgical threshold (e.g. 55 mm) size at which surgery is usually performed. The method of the present invention may be used to identify patients who may benefit from early invasive clinical intervention, for example, even before the patient's aneurysm reaches a surgical threshold (e.g. 55 mm) size. In some embodiments, the clinical intervention may be an invasive clinical intervention, for example, if the size of the abdominal aorta/aortic aneurysm is below a surgical threshold, for instance, from 40 to 55 millimetres. Thus, the method of the present invention can be used to negate unnecessary delay in surgery (as those patients with fast growing AAAs would require surgery in the near future).

The method of the present invention may also be used to guide a decision on a clinical intervention that is non-invasive or cognitive. For example, where the determined risk value of a patient is low, the patient's aneurysm may be slow-growing. Thus, the patient in question need not be monitored highly frequently even, for example, if the patient's aneurysm is relatively close to the threshold size (e.g. 55 mm) at which surgery is usually performed. Accordingly, a schedule e.g. for the patient's future check-ups may be determined at least in part by the determined risk value. Thus, in some embodiments, the method of the present invention may be used to guide a physician's decision as to, for example, the time interval between the patient's follow-up appointment(s). In some embodiments, this surveillance or time interval may be individualised to the patient based on the predicted propensity of future AAA growth.

The aneurysm risk model may be generated based on: protein concentration measurements of at least one patient taken at a first time; and protein concentration measurements of the at least one patient taken at a second time different from the first time.

In certain embodiments, the aneurysm risk model may also generated on the basis of a value representative of a size of the abdominal aorta/aortic aneurysm at the first time and a size of the abdominal aorta/aortic aneurysm artery at the second time.

In some examples, the aneurysm risk model is generated from data collected from a number of patients e.g. in a clinical trial.

The method may also comprise measuring the size of the abdominal aorta/aortic aneurysm of a patient. Any suitable method of measurement may be employed. For example, the maximal anteroposterior diameter (outer-to-outer) of an abdominal aorta/aortic aneurysm may be determined by, for example, by ultrasound scans, computerised tomography, or magnetic resonance imaging.

In one embodiment, the present disclosure provides a method for determining a risk value indicative of predicted growth of an abdominal aortic aneurysm of a patient having an abdominal aortic aneurysm that is less than 55 mm, preferably 30 to 55 mm, more preferably 35 to 55 mm, yet more preferably 40 to 55 mm in size. The method comprises receiving a value representative of or representing a size of the abdominal aorta/aortic aneurysm, wherein the received value is representative of an abdominal aorta/aortic aneurysm that is less than 55 mm, preferably 30 to 55 mm, more preferably 35 to 55 mm, yet more preferably 40 to 55 mm in size. The method also comprises receiving a protein concentration value for at least one protein in a blood sample of the patient. The method further comprises determining a risk value indicative of predicted growth by evaluating the received values with those in an aneurysm risk model. The aneurysm risk model relates to a risk value indicative of predicted growth of an abdominal aortic aneurysm for a given value representative of or representing the size of an abdominal aorta/aortic aneurysm and a given protein concentration value. The given value representative of or representing the size of an abdominal aorta/aortic aneurysm of the risk model may be a value that is representative of or representing an abdominal aorta/aortic aneurysm that is less than 55 mm, preferably 30 to 55 mm, more preferably 35 to 55 mm, yet more preferably 40 to 55 mm in size.

In some examples, the method can be used to determine a risk value indicative of predicted growth of an abdominal aortic aneurysm of a patient having an abdominal aortic aneurysm that is 35 to 55 mm in size, preferably 36 to 53 mm, more preferably 38 to 50 mm in size. The method may involve receiving a value representative of or representing a size of the abdominal aorta/aortic aneurysm, wherein the received value is representative of or representing an abdominal aorta/aortic aneurysm that is 35 to 55 mm in size, preferably 36 to 53 mm, more preferably 38 to 50 mm in size. The given value representative of or representing the size of an abdominal aorta/aortic aneurysm of the risk model may be a value that is representative of or representing an abdominal aorta/aortic aneurysm that is 35 to 55 mm in size, preferably 36 to 53 mm, more preferably 38 to 50 mm in size.

In accordance with a further aspect of the present invention, there is provided an apparatus for determining a risk value indicative of predicted growth of an abdominal aortic aneurysm of a patient. The apparatus comprises a data input to receive a value representative of or representing a size of an abdominal aorta/aortic aneurysm of a patient; and a protein concentration value for at least one protein in a sample of blood of the patient. The apparatus also comprises at least one processor; and a memory comprising an aneurysm risk model relating to a risk value indicative of predicted growth of an abdominal aortic aneurysm for a given value representative of or representing the size of an abdominal aorta/aortic aneurysm and a given blood protein concentration value. The memory also comprises instructions executable by the at least one processor to cause the processor to retrieve from the aneurysm risk model, using the received data inputs, a risk value indicative of predicted growth of an abdominal aortic aneurysm.

In the apparatus described herein, the data input may receive a value representative of or representing a size of the abdominal aorta/aortic aneurysm; and a protein concentration value for at least one protein in the blood of the patient.

In some embodiments, the data input may be a user input device such as a touchscreen interface or a keypad. The data input may be a removable memory connector to receive a removable storage medium to store a value representative of or representing a size of an abdominal aorta/aortic aneurysm of a patient; and a protein concentration value for a blood sample of the patient.

In some embodiments, the apparatus may further comprise a blood sample analysis module in data communication with the data input and configured to analyse one or more predetermined proteins in a blood sample.

Where the apparatus comprises a data memory in data communication with the data input, the data memory may be configured to store one or more of the at least one value representative of or representing a size of an abdominal aorta/aortic aneurysm of a patient; and a blood protein concentration value for at least one protein in a blood sample of the patient. The memory may comprise instructions to cause the processor to read the one or more of the value representative of or representing a size of an abdominal aorta/aortic aneurysm of a patient; and a blood protein concentration value for at least one protein in a blood sample of the patient from the data memory. The memory may further comprise instructions to cause the processor to measure the size of the abdominal aorta/aortic aneurysm.

In yet another aspect, the present invention provides a non-transitory machine-readable storage medium encoded with instructions executable by a processor for determining a risk value indicative of predicted growth of an abdominal aortic aneurysm of a patient. The machine-readable storage medium comprises instructions to receive a value representative of or representing a size of the abdominal aorta/aortic aneurysm; and a blood protein concentration value of the patient. The machine-readable storage medium also comprises instructions to determine the risk value indicative of predicted growth by evaluating the received values with those in an aneurysm risk model, the aneurysm risk model relating to a risk value indicative of predicted growth of an abdominal aortic aneurysm for a given value representative of or representing the size of an abdominal aortic aneurysm and a given blood protein concentration value

The non-transitory machine-readable storage medium may further comprise instructions to receive a value representative of or representing a size of the abdominal aortic aneurysm; and a blood protein concentration value of an artery of the patient.

The non-transitory machine-readable storage medium may further comprise an aneurysm risk model in the form of a look-up table.

In yet a further aspect, there is provided an apparatus for determining an index of aortic aneurysm future growth for a subject. The apparatus comprises a data input for receiving at least one index of blood protein concentration of a subject, at least one processor, and a memory comprising instructions executable by the at least one processor. The instructions are to cause the at least one processor to compare the or each index of blood protein concentration with at least one of a respective reference value. The instructions may further cause the processor to determine the index or risk of aortic aneurysm future growth based on the comparison. The method may determine an index of abdominal aortic aneurysm future growth. It will be understood that the future growth of the aortic aneurysm is a determination of the future progression of the aortic aneurysm.

Thus, there is provided apparatus for determining an index of aneurysm future progression (e.g. growth) for a subject. The apparatus may be used on subjects regardless of knowledge of the presence of an aneurysm in the subject. In many examples, the apparatus may be used on subjects where the presence and size of an aneurysm are already known.

The index of aneurysm future progression may be qualitative or quantitative.

The blood protein concentration is at least one protein selected from Group A proteins, Group B proteins, Group C proteins and Group D proteins as defined below; or at least one protein selected from two, three or all four groups.

In preferred embodiments, the at least one protein is at least one of attractin (UniProt ID 075882), Apolipoprotein A4 (UniProt ID P06727), Complement C8 (UniProt ID P07360) and HSP90AA5P (UniProt ID Q58FG0).

The memory may further comprise instructions to cause the processor to analyse, in vitro using a blood sample analysis module, a sample derived from a blood sample of the subject.

There is also provided a computer-implemented method for determining an index or risk of aneurysm future growth for a subject. The method is implemented on a computer comprising at least one processor and a memory comprising instructions to be executed by the at least one processor. The method comprises: the memory of the computer receiving at least one value of protein concentration in a sample of blood of the subject. The method further comprises the at least one processor comparing the at least one value with at least one respective reference value. The method further comprises the at least one processor determining the index of aneurysm future growth based on the comparison.

The computer-implemented method may further comprise storing index of aneurysm future growth in the memory or a further memory.

The invention extends to a non-transitory machine-readable storage medium encoded with instructions executable by a processor. The machine-readable storage medium comprises instructions to receive at least one value of protein concentration in a sample of blood of the subject. The machine-readable storage medium further comprises instructions to compare the value of protein concentration in a sample of blood of the subject with at least one respective reference value. The machine-readable storage medium further comprises instructions to determine an index of aneurysm future growth based on the comparison.

It will be understood that the present disclosure extends to any of the steps of a method performed by the apparatus disclosed herein.

The present invention also relates to the use of the proteins as defined in Group A, Group B, Group C and Group D, as defined below, as biomarkers for the determination of a risk value or index for future aneurysm growth for a subject.

### Methods

Plasma samples from patients recruited to the study were collected at baseline and at one year from each patient. Plasma samples were also collected before and at 10-12 weeks after surgery from each patient (n=29). Paired aneurysm wall, ILT and omental biopsies were collected intra-operatively during open surgical repair (n=3). In addition to analyses of the tissue, supernatant was obtained from ex vivo culture of these paired tissue samples. Samples were subjected to non-targeted LC-MSMS workflow after trypsin digest, using the Universal method to discover novel proteins. LC-MSMS data was analysed using the Progenesis QI pipeline.

The median AAA size at baseline was 48 mm. 59 patients were prospectively followed for 12 months. The median growth rate of AAA was 3.8%/year (IQR 1.9% to 6.8%).

### Blood Sample Collection

Venous blood samples were collected in tubes containing an anticoagulant such as ethylenediamine tetraacetic acid (EDTA). Plasma samples were centrifuged at room temperature for 12 minutes at 1300 g. Thereafter, the plasma supernatants from the blood were aspirated and centrifuged again for 15 mins at 2500 g. This two stage centrifugation process results in separation of platelet poor plasma. Aliquots of the EDTA plasma were stored at −80° C. for subsequent analysis.

### Tissue Sample Collection

In a designated area of theatre, a pre-prepared trolley housed all the necessary equipment and consumables required for specimen retrieval. Upon entering the abdominal cavity, the surgeon removed a wedge of omentum and an omental artery (OA) was dissected from within it. Some of the omentum was stored in ice-cold RPMI 1640+FCS 5% for the secretome experiments. The remaining omentum and OA were biobanked and some was also fixed using optimum cutting temperature (OCT) solution and paraformaldehyde (PFA) techniques. Prior to the aortic clamp being placed, the surgeon marked the maximal area of the aneurysm. Once the clamps were placed, a cranio-caudal incision was made into the aortic sac, and any residual blood was cleared with suction. The intraluminal thrombus (ILT) was delivered through the aortic incision and on the research table was rinsed thoroughly with 0.9% normal saline to remove any contaminating blood. This was then split up in to pieces, from the luminal and abluminal areas respectively. Fragments were snap frozen for subsequent analysis.

A cranio-caudal aortic anterior aneurysm wall strip from the renal arteries to the iliac bifurcation was trimmed away, spanning, approximately 15 mm in width. A Ligaclip® on the proximal (cranial) aspect denoted the orientation of the specimen. The aortic wall was rinsed through thoroughly with sterile 0.9% normal saline to remove any blood and debris. Any visible fat was trimmed off the adventitia. The specimen was divided in fragments and snap frozen for subsequent analysis.

### Preparation of Tissue Supernatant

Working under sterile aseptic conditions in a tissue culture hood, six pieces of each tissue type (ILT, aortic wall, omentum) were dissected out in 3 mm×3 mm blocks and placed in a 6-well plate (Corning, USA) and resuspended in 2 mL of RPMI 1640+5% FCS. One well was used as a control, following the same sequential washing and media change steps with no tissue in it. The 6-well plates were then placed in to the incubator at 37° C. with 5% CO₂.

Giving the tissue a chance to recover and equilibrate, after an hour they were removed and washed with pre-warmed phosphate-buffered saline (PBS-Lonza) and then serum free RPMI media (Lonza) was added. After 24 hours in the incubator, the plates were removed, and the conditioned media was collected. It was then centrifuged at 3000 g for 10 mins to pellet the remaining cells and cellular debris, and the secretome supernatants were then aspirated and stored at −80° C. in 250 μL aliquots (Cryovial).

Protein Extraction from Plasma/Tissue/Supernatant for Proteomic Analysis

Frozen tissues were placed on a dry ice-chilled Steel BioPulverizer (BioSpec, USA) and the frozen tissues were shattered into fine powder by a sharp blow with a hammer. Broken tissue powder was weighed (20-30 mg) and aliquoted into Beads beater tube kept on dry ice. Pulverized tissues were homogenised in beads-beater tubes containing RIPA lysis buffer (25 mM Tris HCl, pH 7.6, 150 mM NaCl, 1% NP-40, 1% sodium deoxycholate, 0.1% SDS) to make it 20 mg/ml. Tissues were homogenized for 4 times at 6,500 Hz for 40s in a beads-beater (Stretton, UK) with chill on ice during each interval. The samples were centrifuged at 10,000 g for 5 min at 4° C. to remove insoluble tissue debris.

The protein concentration in the homogenates were determined by BCA protein assay (bicinchoninic acid) (Thermo Fisher, UK) and 100 μg of total proteins were reduced by adding 200 mM of dithiothreitol (DTT) (Sigma, Germany) to a final concentration of 5 mM for 30 mins at room temperature. Then, alkylated by adding 200 mM of indole acetic acid (IAA) (Sigma, Germany) to a final concentration of 20 mM and incubate for 30 mins at dark. The samples were top up to 200 μl with 6M urea, 100 mM TrisHCL (tris(hydroxymethyl)aminomethane hydrochloride) pH 8.5. Six hundred microliter of Methanol and 150 μl of Chloroform were added and mixed by brief vortex. Then 450 μl of MilluQ-H2O was added and sample centrifuged for 1 min at 12,000 g. The upper aqueous phase was removed and 450 μl of Methanol were added to samples and centrifuged at 12,000 g for 5 min. The supernatant were removed and pellets resuspended in 50 μl of 6M Urea, 100 mM TrisHCL, pH 8.5. Urea concentration was reduced to less than 1M by adding 250 μl of Mil1Q-H2O. Trypsin was added in 1:30 ratio (trypsin:protein) and digested for overnight at 37° C. The peptides were purified by a SepPak C18 cartridge (Waters, UK), dried by Speed Vac centrifugation, and resuspended in 200 μl of buffer A (2% acetonitrile 0.1% formic acid) for LC-MS/MS analysis.

Plasma samples were selected from the cohort, based on the AAA growth characteristics. 10× fastest and 10× slowest growth were selected. 10 μl of EDTA plasma from each patient's samples was retrieved. The plasma samples were pooled in each group for the analysis (Fast vs Slow). Depletion of the top 12 abundant proteins was performed using the Thermos Top 12 Protein Depletion Spin Column (Catalogue #85164). The depleted samples were then processed through reduction, alkylation, chloroform precipitation and trypsin digest.

Tissue supernatant samples were assessed for their protein content using BCA assay. Volume required for ˜40 μg of protein was retrieved from each sample type for subsequent reduction, alkylation, chloroform precipitation and trypsin digest.

### Mass Spectrometry

For peptide analysis, an uHPLC was coupled to a Hybrid Quadrupole-Orbitrap mass spectrometer (LUMOS Fusion, Thermo Scientific, UK). One microliter of 0.5 μg/μL tryptic digested peptides were injected into the LUMOS for analysis. Peptides were separated by a BEH130 C18 column (1.7 mm×25 cm, Waters) at a flow rate of 250 nL/min. The mobile phases consisted of water with 0.1% formic acid, 5% DMSO (buffer A) and 95% acetonitrile with 5% DMSO, 0.1% formic acid (buffer B). A 60 minutes linear gradient from 3% buffer A to 40% buffer B was used. The peptides were ionised by electro spray ionisation and the 20 most abundant ions per MS scan were fragmented by collision-induced dissociation (CID).

### Data Analysis and Statistics

LC-MS/MS spectra was searched against Uniprot human database (version 2017, 20,205 entries) for peptide homology identification. The Uniprot IDs are given in the results below. Analysis of the dataset was performed using the Progenesis QI software (Nonlinear Dynamics). At least two unique peptides were used for protein quantitation using match between runs. The false discovery rate (FDR) was set to 1% for protein and peptide identification. Label free quantitation (LFQ) intensity data were used for further statistical analysis to compare across the different group of tissues. Differentially expressed proteins in the analysis were defined as proteins presenting a statistical difference across the group (P<0.05).

Statistical analyses were performed in Graphpad Prism Version 8.01. Exploratory data analysis was performed for the initial examination of the dataset. Summary statistics are presented in mean (with SD) or median (with IQR) depending on the normality of distribution. We opted to use non-parametric tests for all comparative analyses (Wilcoxon matched pairs signed rank test, Mann-Whitney test, Kruskal-Wallis test, and Spearman rank correlation), as many variables demonstrated non-Gaussian distributions. No transformation of data was performed.

### Results

### Participant Characteristics

From October 2013, 162 patients with AAAs were recruited (Male n=147; Females n=15). The median AAA diameter was 50 mm (IQR: 40-57 mm). The average age of participants was 75 (+1-7) years old at the time of consent. The majority were ex-smokers (66%) and 19% were current smokers. A history of symptomatic atherosclerotic arterial disease was prevalent in this group (ischaemic heart disease: 40%; peripheral arterial disease: 20%; cerebral vascular disease: 12%). The majority of participants reported a prior diagnosis of arterial hypertension (65%) and hypercholesterolemia (59%). However, these were well controlled by long term medical therapy [anti-hypertensive(s): 71%, statin: 75%, anti-platelet(s): 62%], as reflected by their controlled SBP/DBP (138/79±17/12 mmHg) and overall normal cholesterol profiles [median=3.9 mmol/L (IQR 3.3-4.7), lower than 5.2 mmol/L in 82% of participants] at the time of recruitment. Seventeen percent of the participants reported a history of diabetes mellitus, 19% had chronic respiratory disease, 19% had treated neoplasms, 24% had chronic kidney disease with eGFR<60 (Table 1). There was no correlation between flow mediation vasodilation at baseline with any demographic variables except the AP diameter of AAA.

Experiment 1: Comparison Between Plasma Samples of Those with Fast Vs Slow Growth of AAA in the Future 12 Months

The concentrations of 117 proteins were observed to be significantly different between the plasma samples between those patients showing fast AAA growth between the baseline and one-year analyses and those patients showing slow growth of AAA over the same period. These proteins are listed in Table 1 below and are referred to in the following discussion as Group A, or ‘Fast-v-Slow AAA growth’.

TABLE 1
Group A
Fast-v-Slow AAA Growth

| UNIPROT ID | Description |
|---|---|
| A0A0C4DH31 | Immunoglobulin heavy variable 1-18 OS = Homo sapiens |
| | GN = IGHV1-18 PE = 3 SV = 1 |
| A0A0C4DH68 | Immunoglobulin kappa variable 2-24 OS = Homo sapiens |
| | GN = IGKV2-24 PE = 3 SV = 1 |
| O00533 | Neural cell adhesion molecule L1-like protein OS = Homo sapiens |
| | GN = CHL1 PE = 1 SV = 4 |
| O00555 | Voltage-dependent P/Q-type calcium channel subunit alpha-1A |
| | OS = Homo sapiens GN = CACNA1A PE = 1 SV = 2 |
| O15144 | Actin-related protein 2/3 complex subunit 2 OS = Homo sapiens |
| | GN = ARPC2 PE = 1 SV = 1 |
| O43488 | Aflatoxin B1 aldehyde reductase member 2 OS = Homo sapiens |
| | GN = AKR7A2 PE = 1 SV = 3 |
| O60641 | Clathrin coat assembly protein AP180 OS = Homo sapiens |
| | GN = SNAP91 PE = 1 SV = 2 |
| O75882 | Attractin OS = Homo sapiens GN = ATRN PE = 1 SV = 2 |
| O75891 | Cytosolic 10-formyltetrahydrofolate dehydrogenase OS = Homo |
| | sapiens GN = ALDH1L1 PE = 1 SV = 2 |
| O76013 | Keratin, type I cuticular Ha6 OS = Homo sapiens GN = KRT36 PE = 1 |
| | SV = 1 |
| O95568 | Histidine protein methyltransferase 1 homolog OS = Homo sapiens |
| | GN = METTL18 PE = 1 SV = 1 |
| O95782 | AP-2 complex subunit alpha-1 OS = Homo sapiens GN = AP2A1 PE = 1 |
| | SV = 3 |
| O95810 | Caveolae-associated protein 2 OS = Homo sapiens GN = CAVIN2 |
| | PE = 1 SV = 3 |
| P00390 | Glutathione reductase, mitochondrial OS = Homo sapiens GN = GSR |
| | PE = 1 SV = 2 |
| P00746 | Complement factor D OS = Homo sapiens GN = CFD PE = 1 SV = 5 |
| P00915 | Carbonic anhydrase 1 OS = Homo sapiens GN = CA1 PE = 1 SV = 2 |
| P01034 | Cystatin-C OS = Homo sapiens GN = CST3 PE = 1 SV = 1 |
| P01764 | Immunoglobulin heavy variable 3-23 OS = Homo sapiens |
| | GN = IGHV3-23 PE = 1 SV = 2 |
| P01860 | Immunoglobulin heavy constant gamma 3 OS = Homo sapiens |
| | GN = IGHG3 PE = 1 SV = 2 |
| P01871 | Immunoglobulin heavy constant mu OS = Homo sapiens GN = IGHM |
| | PE = 1 SV = 4 |
| P02655 | Apolipoprotein C-II OS = Homo sapiens GN = APOC2 PE = 1 SV = 1 |
| P02656 | Apolipoprotein C-III OS = Homo sapiens GN = APOC3 PE = 1 SV = 1 |
| P02741 | C-reactive protein OS = Homo sapiens GN = CRP PE = 1 SV = 1 |
| P02771 | Alpha-fetoprotein OS = Homo sapiens GN = AFP PE = 1 SV = 1 |
| P02775 | Platelet basic protein OS = Homo sapiens GN = PPBP PE = 1 SV = 3 |
| P02776 | Platelet factor 4 OS = Homo sapiens GN = PF4 PE = 1 SV = 2 |
| P04271 | Protein S100-B OS = Homo sapiens GN = S100B PE = 1 SV = 2 |
| P05091 | Aldehyde dehydrogenase, mitochondrial OS = Homo sapiens |
| | GN = ALDH2 PE = 1 SV = 2 |
| P06576 | ATP synthase subunit beta, mitochondrial OS = Homo sapiens |
| | GN = ATP5B PE = 1 SV = 3 |
| P06727 | Apolipoprotein A-IV OS = Homo sapiens GN = APOA4 PE = 1 SV = 3 |
| P07099 | Epoxide hydrolase 1 OS = Homo sapiens GN = EPHX1 PE = 1 SV = 1 |
| P07195 | L-lactate dehydrogenase B chain OS = Homo sapiens GN = LDHB |
| | PE = 1 SV = 2 |
| P07360 | Complement component C8 gamma chain OS = Homo sapiens |
| | GN = C8G PE = 1 SV = 3 |
| P07858 | Cathepsin B OS = Homo sapiens GN = CTSB PE = 1 SV = 3 |
| P07988 | Pulmonary surfactant-associated protein B OS = Homo sapiens |
| | GN = SFTPB PE = 1 SV = 3 |
| P08133 | Annexin A6 OS = Homo sapiens GN = ANXA6 PE = 1 SV = 3 |
| P08567 | Pleckstrin OS = Homo sapiens GN = PLEK PE = 1 SV = 3 |
| P08729 | Keratin, type II cytoskeletal 7 OS = Homo sapiens GN = KRT7 PE = 1 |
| | SV = 5 |
| P0DOX6 | Immunoglobulin mu heavy chain OS = Homo sapiens PE = 1 SV = 1 |
| P0DP01 | Immunoglobulin heavy variable 1-8 OS = Homo sapiens GN = IGHV1- |
| | 8 PE = 3 SV = 1 |
| P10643 | Complement component C7 OS = Homo sapiens GN = C7 PE = 1 SV = 2 |
| P10809 | 60 kDa heat shock protein, mitochondrial OS = Homo sapiens |
| | GN = HSPD1 PE = 1 SV = 2 |
| P11142 | Heat shock cognate 71 kDa protein OS = Homo sapiens GN = HSPA8 |
| | PE = 1 SV = 1 |
| P11168 | Solute carrier family 2, facilitated glucose transporter member 2 |
| | OS = Homo sapiens GN = SLC2A2 PE = 1 SV = 1 |
| P11217 | Glycogen phosphorylase, muscle form OS = Homo sapiens |
| | GN = PYGM PE = 1 SV = 6 |
| P11309 | Serine/threonine-protein kinase pim-1 OS = Homo sapiens GN = PIM1 |
| | PE = 1 SV = 3 |
| P13500 | C-C motif chemokine 2 OS = Homo sapiens GN = CCL2 PE = 1 SV = 1 |
| P13645 | Keratin, type I cytoskeletal 10 OS = Homo sapiens GN = KRT10 PE = 1 |
| | SV = 6 |
| P13796 | Plastin-2 OS = Homo sapiens GN = LCP1 PE = 1 SV = 6 |
| P14151 | L-selectin OS = Homo sapiens GN = SELL PE = 1 SV = 2 |
| P15311 | Ezrin OS = Homo sapiens GN = EZR PE = 1 SV = 4 |
| P18085 | ADP-ribosylation factor 4 OS = Homo sapiens GN = ARF4 PE = 1 SV = 3 |
| P20774 | Mimecan OS = Homo sapiens GN = OGN PE = 1 SV = 1 |
| P20851 | C4b-binding protein beta chain OS = Homo sapiens GN = C4BPB |
| | PE = 1 SV = 1 |
| P21810 | Biglycan OS = Homo sapiens GN = BGN PE = 1 SV = 2 |
| P21980 | Protein-glutamine gamma-glutamyltransferase 2 OS = Homo sapiens |
| | GN = TGM2 PE = 1 SV = 2 |
| P23470 | Receptor-type tyrosine-protein phosphatase gamma OS = Homo |
| | sapiens GN = PTPRG PE = 1 SV = 4 |
| P24592 | Insulin-like growth factor-binding protein 6 OS = Homo sapiens |
| | GN = IGFBP6 PE = 1 SV = 1 |
| P27348 | 14-3-3 protein theta OS = Homo sapiens GN = YWHAQ PE = 1 SV = 1 |
| P27797 | Calreticulin OS = Homo sapiens GN = CALR PE = 1 SV = 1 |
| P27816 | Microtubule-associated protein 4 OS = Homo sapiens GN = MAP4 |
| | PE = 1 SV = 3 |
| P27824 | Calnexin OS = Homo sapiens GN = CANX PE = 1 SV = 2 |
| P30086 | Phosphatidylethanolamine-binding protein 1 OS = Homo sapiens |
| | GN = PEBP1 PE = 1 SV = 3 |
| P31948 | Stress-induced-phosphoprotein 1 OS = Homo sapiens GN = STIP1 |
| | PE = 1 SV = 1 |
| P32119 | Peroxiredoxin-2 OS = Homo sapiens GN = PRDX2 PE = 1 SV = 5 |
| P33981 | Dual specificity protein kinase TTK OS = Homo sapiens GN = TTK |
| | PE = 1 SV = 2 |
| P35908 | Keratin, type II cytoskeletal 2 epidermal OS = Homo sapiens |
| | GN = KRT2 PE = 1 SV = 2 |
| P42166 | Lamina-associated polypeptide 2, isoform alpha OS = Homo sapiens |
| | GN = TMPO PE = 1 SV = 2 |
| P46821 | Microtubule-associated protein 1B OS = Homo sapiens GN = MAP1B |
| | PE = 1 SV = 2 |
| P48643 | T-complex protein 1 subunit epsilon OS = Homo sapiens GN = CCT5 |
| | PE = 1 SV = 1 |
| P49458 | Signal recognition particle 9 kDa protein OS = Homo sapiens |
| | GN = SRP9 PE = 1 SV = 2 |
| P50990 | T-complex protein 1 subunit theta OS = Homo sapiens GN = CCT8 |
| | PE = 1 SV = 4 |
| P51884 | Lumican OS = Homo sapiens GN = LUM PE = 1 SV = 2 |
| P54727 | UV excision repair protein RAD23 homolog B OS = Homo sapiens |
| | GN = RAD23B PE = 1 SV = 1 |
| P55884 | Eukaryotic translation initiation factor 3 subunit B OS = Homo sapiens |
| | GN = EIF3B PE = 1 SV = 3 |
| P57721 | Poly(rC)-binding protein 3 OS = Homo sapiens GN = PCBP3 PE = 2 |
| | SV = 2 |
| P61769 | Beta-2-microglobulin OS = Homo sapiens GN = B2M PE = 1 SV = 1 |
| P61916 | Epididymal secretory protein E1 OS = Homo sapiens GN = NPC2 |
| | PE = 1 SV = 1 |
| P62306 | Small nuclear ribonucleoprotein F OS = Homo sapiens GN = SNRPF |
| | PE = 1 SV = 1 |
| P78371 | T-complex protein 1 subunit beta OS = Homo sapiens GN = CCT2 |
| | PE = 1 SV = 4 |
| P81172 | Hepcidin OS = Homo sapiens OX = 9606 GN = HAMP PE = 1 SV = 2 |
| Q04637 | Eukaryotic translation initiation factor 4 gamma 1 OS = Homo sapiens |
| | GN = EIF4G1 PE = 1 SV = 4 |
| Q12906 | Interleukin enhancer-binding factor 3 OS = Homo sapiens GN = ILF3 |
| | PE = 1 SV = 3 |
| Q13093 | Platelet-activating factor acetylhydrolase OS = Homo sapiens |
| | GN = PLA2G7 PE = 1 SV = 1 |
| Q13162 | Peroxiredoxin-4 OS = Homo sapiens GN = PRDX4 PE = 1 SV = 1 |
| Q14019 | Coactosin-like protein OS = Homo sapiens GN = COTL1 PE = 1 SV = 3 |
| Q14204 | Cytoplasmic dynein 1 heavy chain 1 OS = Homo sapiens |
| | GN = DYNC1H1 PE = 1 SV = 5 |
| Q14315 | Filamin-C OS = Homo sapiens GN = FLNC PE = 1 SV = 3 |
| Q15084 | Protein disulfide-isomerase A6 OS = Homo sapiens GN = PDIA6 PE = 1 |
| | SV = 1 |
| Q15847 | Adipogenesis regulatory factor OS = Homo sapiens GN = ADIRF PE = 1 |
| | SV = 1 |
| Q16363 | Laminin subunit alpha-4 OS = Homo sapiens GN = LAMA4 PE = 1 |
| | SV = 4 |
| Q4VNC0 | Probable cation-transporting ATPase 13A5 OS = Homo sapiens |
| | GN = ATP13A5 PE = 2 SV = 1 |
| Q58FG0 | Putative heat shock protein HSP 90-alpha A5 OS = Homo sapiens |
| | GN = HSP90AA5P PE = 2 SV = 1 |
| Q5MJ70 | Speedy protein A OS = Homo sapiens GN = SPDYA PE = 1 SV = 2 |
| Q5VST9 | Obscurin OS = Homo sapiens GN = OBSCN PE = 1 SV = 3 |
| Q6IMN6 | Caprin-2 OS = Homo sapiens GN = CAPRIN2 PE = 1 SV = 1 |
| Q6UX71 | Plexin domain-containing protein 2 OS = Homo sapiens GN = PLXDC2 |
| | PE = 1 SV = 1 |
| Q6ZN28 | Metastasis-associated in colon cancer protein 1 OS = Homo sapiens |
| | GN = MACC1 PE = 1 SV = 2 |
| Q702N8 | Xin actin-binding repeat-containing protein 1 OS = Homo sapiens |
| | GN = XIRP1 PE = 1 SV = 1 |
| Q7Z3D4 | LysM and putative peptidoglycan-binding domain-containing protein |
| | 3 OS = Homo sapiens GN = LYSMD3 PE = 1 SV = 2 |
| Q7Z4W1 | L-xylulose reductase OS = Homo sapiens GN = DCXR PE = 1 SV = 2 |
| Q8N436 | Inactive carboxypeptidase-like protein X2 OS = Homo sapiens |
| | GN = CPXM2 PE = 2 SV = 3 |
| Q8WZ75 | Roundabout homolog 4 OS = Homo sapiens GN = ROBO4 PE = 1 |
| | SV = 1 |
| Q96SN8 | CDK5 regulatory subunit-associated protein 2 OS = Homo sapiens |
| | GN = CDK5RAP2 PE = 1 SV = 5 |
| Q96TC7 | Regulator of microtubule dynamics protein 3 OS = Homo sapiens |
| | GN = RMDN3 PE = 1 SV = 2 |
| Q99497 | Protein/nucleic acid deglycase DJ-1 OS = Homo sapiens GN = PARK7 |
| | PE = 1 SV = 2 |
| Q99879 | Histone H2B type 1-M OS = Homo sapiens GN = HIST 1H2BM PE = 1 |
| | SV = 3 |
| Q99943 | 1-acyl-sn-glycerol-3-phosphate acyltransferase alpha OS = Homo |
| | sapiens GN = AGPAT1 PE = 1 SV = 2 |
| Q9BSJ8 | Extended synaptotagmin-1 OS = Homo sapiens GN = ESYT1 PE = 1 |
| | SV = 1 |
| Q9BZA8 | Protocadherin-11 Y-linked OS = Homo sapiens GN = PCDH11Y PE = 1 |
| | SV = 1 |
| Q9BZK3 | Putative nascent polypeptide-associated complex subunit alpha-like |
| | protein OS = Homo sapiens GN = NACAP1 PE = 5 SV = 1 |
| Q9H361 | Polyadenylate-binding protein 3 OS = Homo sapiens GN = PABPC3 |
| | PE = 1 SV = 2 |
| Q9NQH7 | Probable Xaa-Pro aminopeptidase 3 OS = Homo sapiens |
| | GN = XPNPEP3 PE = 1 SV = 1 |
| Q9NVI1 | Fanconi anemia group I protein OS = Homo sapiens GN = FANCI |
| | PE = 1 SV = 4 |
| Q9UNM6 | 26S proteasome non-ATPase regulatory subunit 13 OS = Homo |
| | sapiens GN = PSMD13 PE = 1 SV = 2 |
| Q9Y5C1 | Angiopoietin-related protein 3 OS = Homo sapiens GN = ANGPTL3 |
| | PE = 1 SV = 1 |
| Q9Y5Z4 | Heme-binding protein 2 OS = Homo sapiens GN = HEBP2 PE = 1 SV = 1 |

Experiment 2: Comparison Between Plasma Samples of Before and After Surgical AAA Repair

The concentrations of 258 proteins were observed to be significantly different between the plasma samples between those before and after surgery to repair the abdominal aortic aneurysms. These proteins are most likely to be released from the thrombus of the AAA, and are thus removed from entering circulation as the thrombus is removed en-bloc during the surgical repair. These proteins are listed in Table 2 below and are referred to in the following discussion as Group B proteins or ‘Before-v-After’ (surgery).

TABLE 2
Group B
Before-v-After

| UNIPROT ID | Description |
|---|---|
| A0A0B4J1V2 | Immunoglobulin heavy variable 2-26 OS = Homo sapiens |
| | GN = IGHV2-26 PE = 3 SV = 1 |
| A0A0B4J2H0 | Immunoglobulin heavy variable 1-69D OS = Homo sapiens |
| | GN = IGHV1-69D PE = 3 SV = 1 |
| A0A0C4DH31 | Immunoglobulin heavy variable 1-18 OS = Homo sapiens |
| | GN = IGHV1-18 PE = 3 SV = 1 |
| A0A0C4DH34 | Immunoglobulin heavy variable 4-28 OS = Homo sapiens |
| | GN = IGHV4-28 PE = 3 SV = 1 |
| A0A0C4DH73 | Immunoglobulin kappa variable 1-12 OS = Homo sapiens |
| | GN = IGKV1-12 PE = 3 SV = 1 |
| A8MTJ3 | Guanine nucleotide-binding protein G(t) subunit alpha-3 |
| | OS = Homo sapiens GN = GNAT3 PE = 2 SV = 2 |
| B5ME19 | Eukaryotic translation initiation factor 3 subunit C-like protein |
| | OS = Homo sapiens GN = EIF3CL PE = 3 SV = 1 |
| B9A064 | Immunoglobulin lambda-like polypeptide 5 OS = Homo sapiens |
| | GN = IGLL5 PE = 2 SV = 2 |
| O00232 | 26S proteasome non-ATPase regulatory subunit 12 OS = Homo |
| | sapiens GN = PSMD12 PE = 1 SV = 3 |
| O00299 | Chloride intracellular channel protein 1 OS = Homo sapiens |
| | GN = CLIC1 PE = 1 SV = 4 |
| O00391 | Sulfhydryl oxidase 1 OS = Homo sapiens GN = QSOX1 PE = 1 SV = 3 |
| O00410 | Importin-5 OS = Homo sapiens GN = IPO5 PE = 1 SV = 4 |
| O00499 | Myc box-dependent-interacting protein 1 OS = Homo sapiens |
| | GN = BIN1 PE = 1 SV = 1 |
| O00555 | Voltage-dependent P/Q-type calcium channel subunit alpha-1A |
| | OS = Homo sapiens GN = CACNA1A PE = 1 SV = 2 |
| O00602 | Ficolin-1 OS = Homo sapiens GN = FCN1 PE = 1 SV = 2 |
| O14786 | Neuropilin-1 OS = Homo sapiens GN = NRP1 PE = 1 SV = 3 |
| O14791 | Apolipoprotein L1 OS = Homo sapiens GN = APOL1 PE = 1 SV = 5 |
| O15061 | Synemin OS = Homo sapiens GN = SYNM PE = 1 SV = 2 |
| O15259 | Nephrocystin-1 OS = Homo sapiens GN = NPHP1 PE = 1 SV = 1 |
| O43390 | Heterogeneous nuclear ribonucleoprotein R OS = Homo sapiens |
| | GN = HNRNPR PE = 1 SV = 1 |
| O43566 | Regulator of G-protein signaling 14 OS = Homo sapiens |
| | GN = RGS14 PE = 1 SV = 4 |
| O43790 | Keratin, type II cuticular Hb6 OS = Homo sapiens GN = KRT86 |
| | PE = 1 SV = 1 |
| O75531 | Barrier-to-autointegration factor OS = Homo sapiens GN = BANF1 |
| | PE = 1 SV = 1 |
| O75636 | Ficolin-3 OS = Homo sapiens GN = FCN3 PE = 1 SV = 2 |
| O75882 | Attractin OS = Homo sapiens GN = ATRN PE = 1 SV = 2 |
| O94856 | Neurofascin OS = Homo sapiens GN = NFASC PE = 1 SV = 4 |
| O94927 | HAUS augmin-like complex subunit 5 OS = Homo sapiens |
| | GN = HAUS5 PE = 1 SV = 2 |
| O94952 | F-box only protein 21 OS = Homo sapiens GN = FBXO21 PE = 2 |
| | SV = 2 |
| O95568 | Histidine protein methyltransferase 1 homolog OS = Homo sapiens |
| | GN = METTL18 PE = 1 SV = 1 |
| P00441 | Superoxide dismutase [Cu—Zn] OS = Homo sapiens GN = SOD1 |
| | PE = 1 SV = 2 |
| P00740 | Coagulation factor IX OS = Homo sapiens GN = F9 PE = 1 SV = 2 |
| P00742 | Coagulation factor X OS = Homo sapiens GN = F10 PE = 1 SV = 2 |
| P00915 | Carbonic anhydrase 1 OS = Homo sapiens GN = CA1 PE = 1 SV = 2 |
| P01624; A0A087WSY6; | Immunoglobulin kappa variable 3-15 OS = Homo sapiens |
| A0A0C4DH55 | GN = IGKV3-15 PE = 1 SV = 2 |
| P01700 | Immunoglobulin lambda variable 1-47 OS = Homo sapiens |
| | GN = IGLV1-47 PE = 1 SV = 2 |
| P01764 | Immunoglobulin heavy variable 3-23 OS = Homo sapiens |
| | GN = IGHV3-23 PE = 1 SV = 2 |
| P01772 | Immunoglobulin heavy variable 3-33 OS = Homo sapiens |
| | GN = IGHV3-33 PE = 1 SV = 2 |
| P01780 | Immunoglobulin heavy variable 3-7 OS = Homo sapiens |
| | GN = IGHV3-7 PE = 1 SV = 2 |
| P01782 | Immunoglobulin heavy variable 3-9 OS = Homo sapiens |
| | GN = IGHV3-9 PE = 1 SV = 2 |
| P02452 | Collagen alpha-1(I) chain OS = Homo sapiens GN = COL1A1 PE = 1 |
| | SV = 5 |
| P02533 | Keratin, type I cytoskeletal 14 OS = Homo sapiens GN = KRT14 |
| | PE = 1 SV = 4 |
| P02671 | Fibrinogen alpha chain OS = Homo sapiens GN = FGA PE = 1 SV = 2 |
| P02748 | Complement component C9 OS = Homo sapiens GN = C9 PE = 1 |
| | SV = 2 |
| P02749 | Beta-2-glycoprotein 1 OS = Homo sapiens GN = APOH PE = 1 SV = 3 |
| P02768 | Serum albumin OS = Homo sapiens GN = ALB PE = 1 SV = 2 |
| P02786 | Transferrin receptor protein 1 OS = Homo sapiens GN = TFRC |
| | PE = 1 SV = 2 |
| P03950 | Angiogenin OS = Homo sapiens GN = ANG PE = 1 SV = 1 |
| P04070 | Vitamin K-dependent protein C OS = Homo sapiens GN = PROC |
| | PE = 1 SV = 1 |
| P04406 | Glyceraldehyde-3-phosphate dehydrogenase OS = Homo sapiens |
| | GN = GAPDH PE = 1 SV = 3 |
| P05106 | Integrin beta-3 OS = Homo sapiens GN = ITGB3 PE = 1 SV = 2 |
| P05109 | Protein S100-A8 OS = Homo sapiens GN = S100A8 PE = 1 SV = 1 |
| P05120 | Plasminogen activator inhibitor 2 OS = Homo sapiens |
| | GN = SERPINB2 PE = 1 SV = 2 |
| P05121 | Plasminogen activator inhibitor 1 OS = Homo sapiens |
| | GN = SERPINE1 PE = 1 SV = 1 |
| P05154 | Plasma serine protease inhibitor OS = Homo sapiens |
| | GN = SERPINA5 PE = 1 SV = 3 |
| P05155 | Plasma protease C1 inhibitor OS = Homo sapiens GN = SERPING1 |
| | PE = 1 SV = 2 |
| P05156 | Complement factor I OS = Homo sapiens GN = CFI PE = 1 SV = 2 |
| P05386 | 60S acidic ribosomal protein P1 OS = Homo sapiens GN = RPLP1 |
| | PE = 1 SV = 1 |
| P05452 | Tetranectin OS = Homo sapiens GN = CLEC3B PE = 1 SV = 3 |
| P05543 | Thyroxine-binding globulin OS = Homo sapiens GN = SERPINA7 |
| | PE = 1 SV = 2 |
| P06276 | Cholinesterase OS = Homo sapiens GN = BCHE PE = 1 SV = 1 |
| P06310 | Immunoglobulin kappa variable 2-30 OS = Homo sapiens |
| | GN = IGKV2-30 PE = 3 SV = 2 |
| P06727 | Apolipoprotein A-IV OS = Homo sapiens GN = APOA4 PE = 1 SV = 3 |
| P06737 | Glycogen phosphorylase, liver form OS = Homo sapiens |
| | GN = PYGL PE = 1 SV = 4 |
| P07099 | Epoxide hydrolase 1 OS = Homo sapiens GN = EPHX1 PE = 1 SV = 1 |
| P07196 | Neurofilament light polypeptide OS = Homo sapiens GN = NEFL |
| | PE = 1 SV = 3 |
| P07225 | Vitamin K-dependent protein S OS = Homo sapiens GN = PROS1 |
| | PE = 1 SV = 1 |
| P07237 | Protein disulfide-isomerase OS = Homo sapiens GN = P4HB PE = 1 |
| | SV = 3 |
| P07360 | Complement component C8 gamma chain OS = Homo sapiens |
| | GN = C8G PE = 1 SV = 3 |
| P07585 | Decorin OS = Homo sapiens GN = DCN PE = 1 SV = 1 |
| P07738 | Bisphosphoglycerate mutase OS = Homo sapiens GN = BPGM |
| | PE = 1 SV = 2 |
| P07741 | Adenine phosphoribosyltransferase OS = Homo sapiens |
| | GN = APRT PE = 1 SV = 2 |
| P08185 | Corticosteroid-binding globulin OS = Homo sapiens |
| | GN = SERPINA6 PE = 1 SV = 1 |
| P08294 | Extracellular superoxide dismutase [Cu—Zn] OS = Homo sapiens |
| | GN = SOD3 PE = 1 SV = 2 |
| P08519 | Apolipoprotein(a) OS = Homo sapiens GN = LPA PE = 1 SV = 1 |
| P08567 | Pleckstrin OS = Homo sapiens GN = PLEK PE = 1 SV = 3 |
| P08571 | Monocyte differentiation antigen CD14 OS = Homo sapiens |
| | GN = CD14 PE = 1 SV = 2 |
| P08729 | Keratin, type II cytoskeletal 7 OS = Homo sapiens GN = KRT7 |
| | PE = 1 SV = 5 |
| P09211 | Glutathione S-transferase P OS = Homo sapiens GN = GSTP1 |
| | PE = 1 SV = 2 |
| P09543 | 2′,3′-cyclic-nucleotide 3′-phosphodiesterase OS = Homo sapiens |
| | GN = CNP PE = 1 SV = 2 |
| P0C0L4 | Complement C4-A OS = Homo sapiens GN = C4A PE = 1 SV = 2 |
| P0DOX5 | Immunoglobulin gamma-1 heavy chain OS = Homo sapiens PE = 1 |
| | SV = 1 |
| P10412 | Histone H1.4 OS = Homo sapiens GN = HIST1H1E PE = 1 SV = 2 |
| P10643 | Complement component C7 OS = Homo sapiens GN = C7 PE = 1 |
| | SV = 2 |
| P10645 | Chromogranin-A OS = Homo sapiens GN = CHGA PE = 1 SV = 7 |
| P10809 | 60 kDa heat shock protein, mitochondrial OS = Homo sapiens |
| | GN = HSPD1 PE = 1 SV = 2 |
| P10909 | Clusterin OS = Homo sapiens GN = CLU PE = 1 SV = 1 |
| P11279 | Lysosome-associated membrane glycoprotein 1 OS = Homo |
| | sapiens GN = LAMP1 PE = 1 SV = 3 |
| P13591 | Neural cell adhesion molecule 1 OS = Homo sapiens GN = NCAM1 |
| | PE = 1 SV = 3 |
| P13611 | Versican core protein OS = Homo sapiens GN = VCAN PE = 1 SV = 3 |
| P13797 | Plastin-3 OS = Homo sapiens GN = PLS3 PE = 1 SV = 4 |
| P14649 | Myosin light chain 6B OS = Homo sapiens GN = MYL6B PE = 1 |
| | SV = 1 |
| P15018 | Leukemia inhibitory factor OS = Homo sapiens GN = LIF PE = 1 |
| | SV = 1 |
| P15090 | Fatty acid-binding protein, adipocyte OS = Homo sapiens |
| | GN = FABP4 PE = 1 SV = 3 |
| P15144 | Aminopeptidase N OS = Homo sapiens GN = ANPEP PE = 1 SV = 4 |
| P15151 | Poliovirus receptor OS = Homo sapiens GN = PVR PE = 1 SV = 2 |
| P15259 | Phosphoglycerate mutase 2 OS = Homo sapiens GN = PGAM2 |
| | PE = 1 SV = 3 |
| P16035 | Metalloproteinase inhibitor 2 OS = Homo sapiens GN = TIMP2 |
| | PE = 1 SV = 2 |
| P17655 | Calpain-2 catalytic subunit OS = Homo sapiens GN = CAPN2 PE = 1 |
| | SV = 6 |
| P17858 | ATP-dependent 6-phosphofructokinase, liver type OS = Homo |
| | sapiens GN = PFKL PE = 1 SV = 6 |
| P17980 | 26S proteasome regulatory subunit 6A OS = Homo sapiens |
| | GN = PSMC3 PE = 1 SV = 3 |
| P19013 | Keratin, type II cytoskeletal 4 OS = Homo sapiens GN = KRT4 |
| | PE = 1 SV = 4 |
| P20339 | Ras-related protein Rab-5A OS = Homo sapiens GN = RAB5A |
| | PE = 1 SV = 2 |
| P20700 | Lamin-B1 OS = Homo sapiens GN = LMNB1 PE = 1 SV = 2 |
| P21810 | Biglycan OS = Homo sapiens GN = BGN PE = 1 SV = 2 |
| P22105 | Tenascin-X OS = Homo sapiens GN = TNXB PE = 1 SV = 4 |
| P22392 | Nucleoside diphosphate kinase B OS = Homo sapiens GN = NME2 |
| | PE = 1 SV = 1 |
| P23142 | Fibulin-1 OS = Homo sapiens GN = FBLN1 PE = 1 SV = 4 |
| P23284 | Peptidyl-prolyl cis-trans isomerase B OS = Homo sapiens |
| | GN = PPIB PE = 1 SV = 2 |
| P23458 | Tyrosine-protein kinase JAK1 OS = Homo sapiens GN = JAK1 |
| | PE = 1 SV = 2 |
| P24821 | Tenascin OS = Homo sapiens GN = TNC PE = 1 SV = 3 |
| P26447 | Protein S100-A4 OS = Homo sapiens GN = S100A4 PE = 1 SV = 1 |
| P27797 | Calreticulin OS = Homo sapiens GN = CALR PE = 1 SV = 1 |
| P30048 | Thioredoxin-dependent peroxide reductase, mitochondrial |
| | OS = Homo sapiens GN = PRDX3 PE = 1 SV = 3 |
| P30740 | Leukocyte elastase inhibitor OS = Homo sapiens GN = SERPINB1 |
| | PE = 1 SV = 1 |
| P31146 | Coronin-1A OS = Homo sapiens GN = CORO1A PE = 1 SV = 4 |
| P31153 | S-adenosylmethionine synthase isoform type-2 OS = Homo |
| | sapiens GN = MAT2A PE = 1 SV = 1 |
| P31323 | cAMP-dependent protein kinase type II-beta regulatory subunit |
| | OS = Homo sapiens GN = PRKAR2B PE = 1 SV = 3 |
| P31943 | Heterogeneous nuclear ribonucleoprotein H OS = Homo sapiens |
| | GN = HNRNPH1 PE = 1 SV = 4 |
| P31946 | 14-3-3 protein beta/alpha OS = Homo sapiens GN = YWHAB PE = 1 |
| | SV = 3 |
| P31948 | Stress-induced-phosphoprotein 1 OS = Homo sapiens GN = STIP1 |
| | PE = 1 SV = 1 |
| P33176 | Kinesin-1 heavy chain OS = Homo sapiens GN = KIF5B PE = 1 |
| | SV = 1 |
| P34931 | Heat shock 70 kDa protein 1-like OS = Homo sapiens |
| | GN = HSPA1L PE = 1 SV = 2 |
| P35442 | Thrombospondin-2 OS = Homo sapiens GN = THBS2 PE = 1 SV = 2 |
| P35542 | Serum amyloid A-4 protein OS = Homo sapiens GN = SAA4 PE = 1 |
| | SV = 2 |
| P35579 | Myosin-9 OS = Homo sapiens GN = MYH9 PE = 1 SV = 4 |
| P35611 | Alpha-adducin OS = Homo sapiens GN = ADD1 PE = 1 SV = 2 |
| P35858 | Insulin-like growth factor-binding protein complex acid labile |
| | subunit OS = Homo sapiens GN = IGFALS PE = 1 SV = 1 |
| P36222 | Chitinase-3-like protein 1 OS = Homo sapiens GN = CHI3L1 PE = 1 |
| | SV = 2 |
| P36269 | Glutathione hydrolase 5 proenzyme OS = Homo sapiens |
| | GN = GGT5 PE = 1 SV = 2 |
| P36955 | Pigment epithelium-derived factor OS = Homo sapiens |
| | GN = SERPINF1 PE = 1 SV = 4 |
| P37837 | Transaldolase OS = Homo sapiens GN = TALDO1 PE = 1 SV = 2 |
| P39059 | Collagen alpha-1(XV) chain OS = Homo sapiens GN = COL15A1 |
| | PE = 1 SV = 2 |
| P40189 | Interleukin-6 receptor subunit beta OS = Homo sapiens GN = IL6ST |
| | PE = 1 SV = 2 |
| P40818 | Ubiquitin carboxyl-terminal hydrolase 8 OS = Homo sapiens |
| | GN = USP8 PE = 1 SV = 1 |
| P41222 | Prostaglandin-H2 D-isomerase OS = Homo sapiens GN = PTGDS |
| | PE = 1 SV = 1 |
| P42330 | Aldo-keto reductase family 1 member C3 OS = Homo sapiens |
| | GN = AKR1C3 PE = 1 SV = 4 |
| P43121 | Cell surface glycoprotein MUC18 OS = Homo sapiens GN = MCAM |
| | PE = 1 SV = 2 |
| P46439 | Glutathione S-transferase Mu 5 OS = Homo sapiens GN = GSTM5 |
| | PE = 1 SV = 3 |
| P46821 | Microtubule-associated protein 1B OS = Homo sapiens |
| | GN = MAP1B PE = 1 SV = 2 |
| P46926 | Glucosamine-6-phosphate isomerase 1 OS = Homo sapiens |
| | GN = GNPDA1 PE = 1 SV = 1 |
| P46939 | Utrophin OS = Homo sapiens GN = UTRN PE = 1 SV = 2 |
| P48735 | Isocitrate dehydrogenase [NADP], mitochondrial OS = Homo |
| | sapiens GN = IDH2 PE = 1 SV = 2 |
| P49327 | Fatty acid synthase OS = Homo sapiens GN = FASN PE = 1 SV = 3 |
| P49336 | Cyclin-dependent kinase 8 OS = Homo sapiens GN = CDK8 PE = 1 |
| | SV = 1 |
| P51153 | Ras-related protein Rab-13 OS = Homo sapiens GN = RAB13 PE = 1 |
| | SV = 1 |
| P51884 | Lumican OS = Homo sapiens GN = LUM PE = 1 SV = 2 |
| P52209 | 6-phosphogluconate dehydrogenase, decarboxylating OS = Homo |
| | sapiens GN = PGD PE = 1 SV = 3 |
| P52789 | Hexokinase-2 OS = Homo sapiens GN = HK2 PE = 1 SV = 2 |
| P53365 | Arfaptin-2 OS = Homo sapiens GN = ARFIP2 PE = 1 SV = 1 |
| P53621 | Coatomer subunit alpha OS = Homo sapiens GN = COPA PE = 1 |
| | SV = 2 |
| P54802 | Alpha-N-acetylglucosaminidase OS = Homo sapiens GN = NAGLU |
| | PE = 1 SV = 2 |
| P55058 | Phospholipid transfer protein OS = Homo sapiens GN = PLTP PE = 1 |
| | SV = 1 |
| P55072 | Transitional endoplasmic reticulum ATPase OS = Homo sapiens |
| | GN = VCP PE = 1 SV = 4 |
| P57721 | Poly(rC)-binding protein 3 OS = Homo sapiens GN = PCBP3 PE = 2 |
| | SV = 2 |
| P60981 | Destrin OS = Homo sapiens GN = DSTN PE = 1 SV = 3 |
| P61077 | Ubiquitin-conjugating enzyme E2 D3 OS = Homo sapiens |
| | GN = UBE2D3 PE = 1 SV = 1 |
| P61604 | 10 kDa heat shock protein, mitochondrial OS = Homo sapiens |
| | GN = HSPE1 PE = 1 SV = 2 |
| P61626 | Lysozyme C OS = Homo sapiens GN = LYZ PE = 1 SV = 1 |
| P61916 | Epididymal secretory protein E1 OS = Homo sapiens GN = NPC2 |
| | PE = 1 SV = 1 |
| P62195 | 26S proteasome regulatory subunit 8 OS = Homo sapiens |
| | GN = PSMC5 PE = 1 SV = 1 |
| P62805 | Histone H4 OS = Homo sapiens GN = HIST1H4A PE = 1 SV = 2 |
| P62993 | Growth factor receptor-bound protein 2 OS = Homo sapiens |
| | GN = GRB2 PE = 1 SV = 1 |
| P63244 | Receptor of activated protein C kinase 1 OS = Homo sapiens |
| | GN = RACK1 PE = 1 SV = 3 |
| P68363 | Tubulin alpha-1B chain OS = Homo sapiens GN = TUBA1B PE = 1 |
| | SV = 1 |
| P78386 | Keratin, type II cuticular Hb5 OS = Homo sapiens GN = KRT85 |
| | PE = 1 SV = 1 |
| P98160 | Basement membrane-specific heparan sulfate proteoglycan core |
| | protein OS = Homo sapiens GN = HSPG2 PE = 1 SV = 4 |
| P99999 | Cytochrome c OS = Homo sapiens GN = CYCS PE = 1 SV = 2 |
| Q00610 | Clathrin heavy chain 1 OS = Homo sapiens GN = CLTC PE = 1 SV = 5 |
| Q00765 | Receptor expression-enhancing protein 5 OS = Homo sapiens |
| | GN = REEP5 PE = 1 SV = 3 |
| Q01459 | Di-N-acetylchitobiase OS = Homo sapiens GN = CTBS PE = 1 SV = 1 |
| Q01546 | Keratin, type II cytoskeletal 2 oral OS = Homo sapiens GN = KRT76 |
| | PE = 1 SV = 2 |
| Q04760 | Lactoylglutathione lyase OS = Homo sapiens GN = GLO1 PE = 1 |
| | SV = 4 |
| Q05707 | Collagen alpha-1 (XIV) chain OS = Homo sapiens GN = COL14A1 |
| | PE = 1 SV = 3 |
| Q08043 | Alpha-actinin-3 OS = Homo sapiens GN = ACTN3 PE = 1 SV = 2 |
| Q10588 | ADP-ribosyl cyclase/cyclic ADP-ribose hydrolase 2 OS = Homo |
| | sapiens GN = BST1 PE = 1 SV = 2 |
| Q12905 | Interleukin enhancer-binding factor 2 OS = Homo sapiens |
| | GN = ILF2 PE = 1 SV = 2 |
| Q12906 | Interleukin enhancer-binding factor 3 OS = Homo sapiens |
| | GN = ILF3 PE = 1 SV = 3 |
| Q12931 | Heat shock protein 75 kDa, mitochondrial OS = Homo sapiens |
| | GN = TRAP1 PE = 1 SV = 3 |
| Q13093 | Platelet-activating factor acetylhydrolase OS = Homo sapiens |
| | GN = PLA2G7 PE = 1 SV = 1 |
| Q13404 | Ubiquitin-conjugating enzyme E2 variant 1 OS = Homo sapiens |
| | GN = UBE2V1 PE = 1 SV = 2 |
| Q13557 | Calcium/calmodulin-dependent protein kinase type II subunit |
| | delta OS = Homo sapiens GN = CAMK2D PE = 1 SV = 3 |
| Q13813 | Spectrin alpha chain, non-erythrocytic 1 OS = Homo sapiens |
| | GN = SPTAN1 PE = 1 SV = 3 |
| Q14112 | Nidogen-2 OS = Homo sapiens GN = NID2 PE = 1 SV = 3 |
| Q14152 | Eukaryotic translation initiation factor 3 subunit A OS = Homo |
| | sapiens GN = EIF3A PE = 1 SV = 1 |
| Q14195 | Dihydropyrimidinase-related protein 3 OS = Homo sapiens |
| | GN = DPYSL3 PE = 1 SV = 1 |
| Q14204 | Cytoplasmic dynein 1 heavy chain 1 OS = Homo sapiens |
| | GN = DYNC1H1 PE = 1 SV = 5 |
| Q14515 | SPARC-like protein 1 OS = Homo sapiens GN = SPARCL1 PE = 1 |
| | SV = 2 |
| Q14974 | Importin subunit beta-1 OS = Homo sapiens GN = KPNB1 PE = 1 |
| | SV = 2 |
| Q15063 | Periostin OS = Homo sapiens GN = POSTN PE = 1 SV = 2 |
| Q15166 | Serum paraoxonase/lactonase 3 OS = Homo sapiens GN = PON3 |
| | PE = 1 SV = 3 |
| Q15819 | Ubiquitin-conjugating enzyme E2 variant 2 OS = Homo sapiens |
| | GN = UBE2V2 PE = 1 SV = 4 |
| Q15847 | Adipogenesis regulatory factor OS = Homo sapiens GN = ADIRF |
| | PE = 1 SV = 1 |
| Q16181 | Septin-7 OS = Homo sapiens GN = SEPT7 PE = 1 SV = 2 |
| Q460N5 | Poly [ADP-ribose] polymerase 14 OS = Homo sapiens |
| | GN = PARP14 PE = 1 SV = 3 |
| Q4VNC0 | Probable cation-transporting ATPase 13A5 OS = Homo sapiens |
| | GN = ATP13A5 PE = 2 SV = 1 |
| Q58FG0 | Putative heat shock protein HSP 90-alpha A5 OS = Homo sapiens |
| | GN = HSP90AA5P PE = 2 SV = 1 |
| Q5VTT5 | Myomesin-3 OS = Homo sapiens GN = MYOM3 PE = 1 SV = 1 |
| Q5VUJ5 | Putative Arf-GAP with GTPase, ANK repeat and PH domain- |
| | containing protein 7 OS = Homo sapiens GN = AGAP7P PE = 5 |
| | SV = 1 |
| Q68DU8 | BTB/POZ domain-containing protein KCTD16 OS = Homo sapiens |
| | GN = KCTD16 PE = 1 SV = 1 |
| Q6EMK4 | Vasorin OS = Homo sapiens GN = VASN PE = 1 SV = 1 |
| Q6UVK1 | Chondroitin sulfate proteoglycan 4 OS = Homo sapiens |
| | GN = CSPG4 PE = 1 SV = 2 |
| Q6YHK3 | CD109 antigen OS = Homo sapiens GN = CD109 PE = 1 SV = 2 |
| Q6ZMV7 | Leucine-, glutamate- and lysine-rich protein 1 OS = Homo sapiens |
| | GN = LEKR1 PE = 2 SV = 2 |
| Q7Z3Y7 | Keratin, type I cytoskeletal 28 OS = Homo sapiens GN = KRT28 |
| | PE = 1 SV = 2 |
| Q86VB7 | Scavenger receptor cysteine-rich type 1 protein M130 OS = Homo |
| | sapiens GN = CD163 PE = 1 SV = 2 |
| Q8IUX7 | Adipocyte enhancer-binding protein 1 OS = Homo sapiens |
| | GN = AEBP1 PE = 1 SV = 1 |
| Q8IZF3 | Adhesion G protein-coupled receptor F4 OS = Homo sapiens |
| | GN = ADGRF4 PE = 2 SV = 3 |
| Q8NBS9 | Thioredoxin domain-containing protein 5 OS = Homo sapiens |
| | GN = TXNDC5 PE = 1 SV = 2 |
| Q8NHM5 | Lysine-specific demethylase 2B OS = Homo sapiens GN = KDM2B |
| | PE = 1 SV = 1 |
| Q8TEP8 | Centrosomal protein of 192 kDa OS = Homo sapiens GN = CEP192 |
| | PE = 1 SV = 2 |
| Q8WXW3 | Progesterone-induced-blocking factor 1 OS = Homo sapiens |
| | GN = PIBF1 PE = 1 SV = 2 |
| Q8WZ75 | Roundabout homolog 4 OS = Homo sapiens GN = ROBO4 PE = 1 |
| | SV = 1 |
| Q969G5 | Caveolae-associated protein 3 OS = Homo sapiens GN = CAVIN3 |
| | PE = 1 SV = 3 |
| Q969H8 | Myeloid-derived growth factor OS = Homo sapiens GN = MYDGF |
| | PE = 1 SV = 1 |
| Q96A00 | Protein phosphatase 1 regulatory subunit 14A OS = Homo sapiens |
| | GN = PPP1R14A PE = 1 SV = 1 |
| Q96IU4 | Protein ABHD14B OS = Homo sapiens GN = ABHD14B PE = 1 |
| | SV = 1 |
| Q96S96 | Phosphatidylethanolamine-binding protein 4 OS = Homo sapiens |
| | GN = PEBP4 PE = 1 SV = 3 |
| Q96SN8 | CDK5 regulatory subunit-associated protein 2 OS = Homo sapiens |
| | GN = CDK5RAP2 PE = 1 SV = 5 |
| Q96T58 | Msx2-interacting protein OS = Homo sapiens GN = SPEN PE = 1 |
| | SV = 1 |
| Q96TC7 | Regulator of microtubule dynamics protein 3 OS = Homo sapiens |
| | GN = RMDN3 PE = 1 SV = 2 |
| Q99456 | Keratin, type I cytoskeletal 12 OS = Homo sapiens GN = KRT12 |
| | PE = 1 SV = 1 |
| Q99497 | Protein/nucleic acid deglycase DJ-1 OS = Homo sapiens |
| | GN = PARK7 PE = 1 SV = 2 |
| Q99542 | Matrix metalloproteinase-19 OS = Homo sapiens GN = MMP19 |
| | PE = 1 SV = 1 |
| Q99715 | Collagen alpha-1 (XII) chain OS = Homo sapiens GN = COL12A1 |
| | PE = 1 SV = 2 |
| Q9BTV5 | Fibronectin type III and SPRY domain-containing protein 1 |
| | OS = Homo sapiens GN = FSD1 PE = 1 SV = 1 |
| Q9BWP8 | Collectin-11 OS = Homo sapiens GN = COLEC11 PE = 1 SV = 1 |
| Q9BYX7 | Putative beta-actin-like protein 3 OS = Homo sapiens |
| | GN = POTEKP PE = 5 SV = 1 |
| Q9BZA8 | Protocadherin-11 Y-linked OS = Homo sapiens GN = PCDH11Y |
| | PE = 1 SV = 1 |
| Q9GZT8 | NIF3-like protein 1 OS = Homo sapiens GN = NIF3L1 PE = 1 SV = 2 |
| Q9H0W9 | Ester hydrolase C11orf54 OS = Homo sapiens GN = C11orf54 |
| | PE = 1 SV = 1 |
| Q9H254 | Spectrin beta chain, non-erythrocytic 4 OS = Homo sapiens |
| | GN = SPTBN4 PE = 1 SV = 2 |
| Q9H2G4 | Testis-specific Y-encoded-like protein 2 OS = Homo sapiens |
| | GN = TSPYL2 PE = 1 SV = 1 |
| Q9H361 | Polyadenylate-binding protein 3 OS = Homo sapiens GN = PABPC3 |
| | PE = 1 SV = 2 |
| Q9H4A9 | Dipeptidase 2 OS = Homo sapiens GN = DPEP2 PE = 1 SV = 2 |
| Q9H521 | Putative uncharacterized protein LOC645739 OS = Homo sapiens |
| | PE = 5 SV = 1 |
| Q9H6S3 | Epidermal growth factor receptor kinase substrate 8-like protein 2 |
| | OS = Homo sapiens GN = EPS8L2 PE = 1 SV = 2 |
| Q9H8L6 | Multimerin-2 OS = Homo sapiens GN = MMRN2 PE = 1 SV = 2 |
| Q9HBM1 | Kinetochore protein Spc25 OS = Homo sapiens GN = SPC25 PE = 1 |
| | SV = 1 |
| Q9NP74 | Palmdelphin OS = Homo sapiens GN = PALMD PE = 1 SV = 1 |
| Q9NPH3 | Interleukin-1 receptor accessory protein OS = Homo sapiens |
| | GN = IL1RAP PE = 1 SV = 2 |
| Q9NQH7 | Probable Xaa-Pro aminopeptidase 3 OS = Homo sapiens |
| | GN = XPNPEP3 PE = 1 SV = 1 |
| Q9NQR4 | Omega-amidase NIT2 OS = Homo sapiens GN = NIT2 PE = 1 SV = 1 |
| Q9NR19 | Acetyl-coenzyme A synthetase, cytoplasmic OS = Homo sapiens |
| | GN = ACSS2 PE = 1 SV = 1 |
| Q9NRF8 | CTP synthase 2 OS = Homo sapiens GN = CTPS2 PE = 1 SV = 1 |
| Q9P2E9 | Ribosome-binding protein 1 OS = Homo sapiens GN = RRBP1 |
| | PE = 1 SV = 4 |
| Q9P2K8 | eIF-2-alpha kinase GCN2 OS = Homo sapiens GN = EIF2AK4 |
| | PE = 1 SV = 3 |
| Q9UBW8 | COP9 signalosome complex subunit 7a OS = Homo sapiens |
| | GN = COPS7A PE = 1 SV = 1 |
| Q9UJ70 | N-acetyl-D-glucosamine kinase OS = Homo sapiens GN = NAGK |
| | PE = 1 SV = 4 |
| Q9UJV3 | Probable E3 ubiquitin-protein ligase MID2 OS = Homo sapiens |
| | GN = MID2 PE = 1 SV = 3 |
| Q9UKU6 | Thyrotropin-releasing hormone-degrading ectoenzyme |
| | OS = Homo sapiens GN = TRHDE PE = 2 SV = 1 |
| Q9ULJ6 | Zinc finger MIZ domain-containing protein 1 OS = Homo sapiens |
| | GN = ZMIZ1 PE = 1 SV = 3 |
| Q9ULK0 | Glutamate receptor ionotropic, delta-1 OS = Homo sapiens |
| | GN = GRID1 PE = 2 SV = 2 |
| Q9UNW1 | Multiple inositol polyphosphate phosphatase 1 OS = Homo |
| | sapiens GN = MINPP1 PE = 1 SV = 1 |
| Q9Y2J2 | Band 4.1-like protein 3 OS = Homo sapiens GN = EPB41L3 PE = 1 |
| | SV = 2 |
| Q9Y2Q5 | Ragulator complex protein LAMTOR2 OS = Homo sapiens |
| | GN = LAMTOR2 PE = 1 SV = 1 |
| Q9Y4F1 | FERM, ARHGEF and pleckstrin domain-containing protein 1 |
| | OS = Homo sapiens GN = FARP1 PE = 1 SV = 1 |
| Q9Y4G2 | Pleckstrin homology domain-containing family M member 1 |
| | OS = Homo sapiens GN = PLEKHM1 PE = 1 SV = 3 |
| Q9Y4G6 | Talin-2 OS = Homo sapiens GN = TLN2 PE = 1 SV = 4 |

Experiment 3: Comparison Between Thrombus Tissue Samples Vs Controls

The abundance of 254 proteins was observed to be significantly higher in the thrombus tissue/ILT tissue compared with control (non-thrombus) tissue. These proteins are listed in Table 3 below and referred to in the following discussion as Group C proteins or ‘Thrombus tissue’.

TABLE 3
Group C
Thrombus Tissue

| UNIPROT ID | Description |
|---|---|
| A0A075B6J9 | Immunoglobulin lambda variable 2-18 OS = Homo sapiens GN = IGLV2- |
| | 18 PE = 3 SV = 2 |
| A0A075B6K5 | Immunoglobulin lambda variable 3-9 OS = Homo sapiens GN = IGLV3-9 |
| | PE = 3 SV = 1 |
| A0A075B6P5 | Immunoglobulin kappa variable 2-28 OS = Homo sapiens GN = IGKV2- |
| | 28 PE = 3 SV = 1 |
| A0A075B6S5 | Immunoglobulin kappa variable 1-27 OS = Homo sapiens GN = IGKV1- |
| | 27 PE = 3 SV = 1 |
| A0A0C4DH67 | Immunoglobulin kappa variable 1-8 OS = Homo sapiens GN = IGKV1-8 |
| | PE = 3 SV = 1 |
| A4UGR9 | Xin actin-binding repeat-containing protein 2 OS = Homo sapiens |
| | GN = XIRP2 PE = 1 SV = 2 |
| A6NFK2 | Glutaredoxin domain-containing cysteine-rich protein 2 OS = Homo |
| | sapiens GN = GRXCR2 PE = 3 SV = 1 |
| E9PAV3 | Nascent polypeptide-associated complex subunit alpha, muscle- |
| | specific form OS = Homo sapiens GN = NACA PE = 1 SV = 1 |
| O00161 | Synaptosomal-associated protein 23 OS = Homo sapiens |
| | GN = SNAP23 PE = 1 SV = 1 |
| O00170 | AH receptor-interacting protein OS = Homo sapiens GN = AIP PE = 1 |
| | SV = 2 |
| O00423 | Echinoderm microtubule-associated protein-like 1 OS = Homo sapiens |
| | GN = EML1 PE = 1 SV = 3 |
| O00429 | Dynamin-1-like protein OS = Homo sapiens GN = DNM1L PE = 1 SV = 2 |
| O14556 | Glyceraldehyde-3-phosphate dehydrogenase, testis-specific |
| | OS = Homo sapiens GN = GAPDHS PE = 1 SV = 2 |
| O14980 | Exportin-1 OS = Homo sapiens GN = XPO1 PE = 1 SV = 1 |
| O43491 | Band 4.1-like protein 2 OS = Homo sapiens GN = EPB41L2 PE = 1 SV = 1 |
| O43681 | ATPase ASNA1 OS = Homo sapiens GN = ASNA1 PE = 1 SV = 2 |
| O43865 | S-adenosylhomocysteine hydrolase-like protein 1 OS = Homo sapiens |
| | GN = AHCYL1 PE = 1 SV = 2 |
| O60669 | Monocarboxylate transporter 2 OS = Homo sapiens GN = SLC16A7 |
| | PE = 1 SV = 2 |
| O60716 | Catenin delta-1 OS = Homo sapiens GN = CTNND1 PE = 1 SV = 1 |
| O75348 | V-type proton ATPase subunit G 1 OS = Homo sapiens |
| | GN = ATP6V1G1 PE = 1 SV = 3 |
| O75636 | Ficolin-3 OS = Homo sapiens GN = FCN3 PE = 1 SV = 2 |
| O75882 | Attractin OS = Homo sapiens GN = ATRN PE = 1 SV = 2 |
| O94769 | Extracellular matrix protein 2 OS = Homo sapiens GN = ECM2 PE = 2 |
| | SV = 1 |
| O94826 | Mitochondrial import receptor subunit TOM70 OS = Homo sapiens |
| | GN = TOMM70 PE = 1 SV = 1 |
| O94911 | ATP-binding cassette sub-family A member 8 OS = Homo sapiens |
| | GN = ABCA8 PE = 1 SV = 3 |
| O95239 | Chromosome-associated kinesin KIF4A OS = Homo sapiens |
| | GN = KIF4A PE = 1 SV = 3 |
| O95373 | Importin-7 OS = Homo sapiens GN = IPO7 PE = 1 SV = 1 |
| O95445 | Apolipoprotein M OS = Homo sapiens GN = APOM PE = 1 SV = 2 |
| O95718 | Steroid hormone receptor ERR2 OS = Homo sapiens GN = ESRRB |
| | PE = 1 SV = 3 |
| P00450 | Ceruloplasmin OS = Homo sapiens GN = CP PE = 1 SV = 1 |
| P00734 | Prothrombin OS = Homo sapiens GN = F2 PE = 1 SV = 2 |
| P00736 | Complement C1r subcomponent OS = Homo sapiens GN = C1R PE = 1 |
| | SV = 2 |
| P00738 | Haptoglobin OS = Homo sapiens GN = HP PE = 1 SV = 1 |
| P00739 | Haptoglobin-related protein OS = Homo sapiens GN = HPR PE = 2 SV = 2 |
| P00747 | Plasminogen OS = Homo sapiens GN = PLG PE = 1 SV = 2 |
| P00748 | Coagulation factor XII OS = Homo sapiens GN = F12 PE = 1 SV = 3 |
| P00751 | Complement factor B OS = Homo sapiens GN = CFB PE = 1 SV = 2 |
| P01008 | Antithrombin-III OS = Homo sapiens GN = SERPINC1 PE = 1 SV = 1 |
| P01009 | Alpha-1-antitrypsin OS = Homo sapiens GN = SERPINA1 PE = 1 SV = 3 |
| P01011 | Alpha-1-antichymotrypsin OS = Homo sapiens GN = SERPINA3 PE = 1 |
| | SV = 2 |
| P01023 | Alpha-2-macroglobulin OS = Homo sapiens GN = A2M PE = 1 SV = 3 |
| P01024 | Complement C3 OS = Homo sapiens GN = C3 PE = 1 SV = 2 |
| P01031 | Complement C5 OS = Homo sapiens GN = C5 PE = 1 SV = 4 |
| P01033 | Metalloproteinase inhibitor 1 OS = Homo sapiens GN = TIMP1 PE = 1 |
| | SV = 1 |
| P01042 | Kininogen-1 OS = Homo sapiens GN = KNG1 PE = 1 SV = 2 |
| P01624 | Immunoglobulin kappa variable 3-15 OS = Homo sapiens GN = IGKV3- |
| | 15 PE = 1 SV = 2 |
| P01704 | Immunoglobulin lambda variable 2-14 OS = Homo sapiens GN = IGLV2- |
| | 14 PE = 1 SV = 2 |
| P01764 | Immunoglobulin heavy variable 3-23 OS = Homo sapiens GN = IGHV3- |
| | 23 PE = 1 SV = 2 |
| P01871 | Immunoglobulin heavy constant mu OS = Homo sapiens GN = IGHM |
| | PE = 1 SV = 4 |
| P01876 | Immunoglobulin heavy constant alpha 1 OS = Homo sapiens |
| | GN = IGHA1 PE = 1 SV = 2 |
| P01911 | HLA class II histocompatibility antigen, DRB1-15 beta chain |
| | OS = Homo sapiens GN = HLA-DRB1 PE = 1 SV = 2 |
| P02647 | Apolipoprotein A-I OS = Homo sapiens GN = APOA1 PE = 1 SV = 1 |
| P02649 | Apolipoprotein E OS = Homo sapiens GN = APOE PE = 1 SV = 1 |
| P02652 | Apolipoprotein A-II OS = Homo sapiens GN = APOA2 PE = 1 SV = 1 |
| P02654 | Apolipoprotein C-I OS = Homo sapiens GN = APOC1 PE = 1 SV = 1 |
| P02671 | Fibrinogen alpha chain OS = Homo sapiens GN = FGA PE = 1 SV = 2 |
| P02675 | Fibrinogen beta chain OS = Homo sapiens GN = FGB PE = 1 SV = 2 |
| P02679 | Fibrinogen gamma chain OS = Homo sapiens GN = FGG PE = 1 SV = 3 |
| P02730 | Band 3 anion transport protein OS = Homo sapiens GN = SLC4A1 PE = 1 |
| | SV = 3 |
| P02743 | Serum amyloid P-component OS = Homo sapiens GN = APCS PE = 1 |
| | SV = 2 |
| P02745 | Complement C1q subcomponent subunit A OS = Homo sapiens |
| | GN = C1QA PE = 1 SV = 2 |
| P02747 | Complement C1q subcomponent subunit C OS = Homo sapiens |
| | GN = C1QC PE = 1 SV = 3 |
| P02748 | Complement component C9 OS = Homo sapiens GN = C9 PE = 1 SV = 2 |
| P02749 | Beta-2-glycoprotein 1 OS = Homo sapiens GN = APOH PE = 1 SV = 3 |
| P02750 | Leucine-rich alpha-2-glycoprotein OS = Homo sapiens GN = LRG1 |
| | PE = 1 SV = 2 |
| P02751 | Fibronectin OS = Homo sapiens GN = FN1 PE = 1 SV = 4 |
| P02753 | Retinol-binding protein 4 OS = Homo sapiens GN = RBP4 PE = 1 SV = 3 |
| P02768 | Serum albumin OS = Homo sapiens GN = ALB PE = 1 SV = 2 |
| P02774 | Vitamin D-binding protein OS = Homo sapiens GN = GC PE = 1 SV = 1 |
| P02776 | Platelet factor 4 OS = Homo sapiens GN = PF4 PE = 1 SV = 2 |
| P02790 | Hemopexin OS = Homo sapiens GN = HPX PE = 1 SV = 2 |
| P02792 | Ferritin light chain OS = Homo sapiens GN = FTL PE = 1 SV = 2 |
| P03952 | Plasma kallikrein OS = Homo sapiens GN = KLKB1 PE = 1 SV = 1 |
| P03999 | Short-wave-sensitive opsin 1 OS = Homo sapiens GN = OPN1SW PE = 1 |
| | SV = 1 |
| P04004 | Vitronectin OS = Homo sapiens GN = VTN PE = 1 SV = 1 |
| P04114 | Apolipoprotein B-100 OS = Homo sapiens GN = APOB PE = 1 SV = 2 |
| P04196 | Histidine-rich glycoprotein OS = Homo sapiens GN = HRG PE = 1 SV = 1 |
| P04217 | Alpha-1 B-glycoprotein OS = Homo sapiens GN = A1BG PE = 1 SV = 4 |
| P04278 | Sex hormone-binding globulin OS = Homo sapiens GN = SHBG PE = 1 |
| | SV = 2 |
| P04430 | Immunoglobulin kappa variable 1-16 OS = Homo sapiens GN = IGKV1- |
| | 16 PE = 1 SV = 2 |
| P05106 | Integrin beta-3 OS = Homo sapiens GN = ITGB3 PE = 1 SV = 2 |
| P05121 | Plasminogen activator inhibitor 1 OS = Homo sapiens GN = SERPINE1 |
| | PE = 1 SV = 1 |
| P05154 | Plasma serine protease inhibitor OS = Homo sapiens GN = SERPINA5 |
| | PE = 1 SV = 3 |
| P05155 | Plasma protease C1 inhibitor OS = Homo sapiens GN = SERPING1 |
| | PE = 1 SV = 2 |
| P05455 | Lupus La protein OS = Homo sapiens GN = SSB PE = 1 SV = 2 |
| P05546 | Heparin cofactor 2 OS = Homo sapiens GN = SERPIND1 PE = 1 SV = 3 |
| P06276 | Cholinesterase OS = Homo sapiens GN = BCHE PE = 1 SV = 1 |
| P06312 | Immunoglobulin kappa variable 4-1 OS = Homo sapiens GN = IGKV4-1 |
| | PE = 1 SV = 1 |
| P06396 | Gelsolin OS = Homo sapiens GN = GSN PE = 1 SV = 1 |
| P06727 | Apolipoprotein A-IV OS = Homo sapiens GN = APOA4 PE = 1 SV = 3 |
| P07358 | Complement component C8 beta chain OS = Homo sapiens GN = C8B |
| | PE = 1 SV = 3 |
| P07360 | Complement component C8 gamma chain OS = Homo sapiens |
| | GN = C8G PE = 1 SV = 3 |
| P07996 | Thrombospondin-1 OS = Homo sapiens GN = THBS1 PE = 1 SV = 2 |
| P08185 | Corticosteroid-binding globulin OS = Homo sapiens GN = SERPINA6 |
| | PE = 1 SV = 1 |
| P08236 | Beta-glucuronidase OS = Homo sapiens GN = GUSB PE = 1 SV = 2 |
| P08238 | Heat shock protein HSP 90-beta OS = Homo sapiens GN = HSP90AB1 |
| | PE = 1 SV = 4 |
| P08246 | Neutrophil elastase OS = Homo sapiens GN = ELANE PE = 1 SV = 1 |
| P08571 | Monocyte differentiation antigen CD14 OS = Homo sapiens GN = CD14 |
| | PE = 1 SV = 2 |
| P08603 | Complement factor H OS = Homo sapiens GN = CFH PE = 1 SV = 4 |
| P09104 | Gamma-enolase OS = Homo sapiens GN = ENO2 PE = 1 SV = 3 |
| P09326 | CD48 antigen OS = Homo sapiens GN = CD48 PE = 1 SV = 2 |
| P09488 | Glutathione S-transferase Mu 1 OS = Homo sapiens GN = GSTM1 |
| | PE = 1 SV = 3 |
| P0DOX6 | Immunoglobulin mu heavy chain OS = Homo sapiens PE = 1 SV = 1 |
| P0DOX7 | Immunoglobulin kappa light chain OS = Homo sapiens PE = 1 SV = 1 |
| P0DP01 | Immunoglobulin heavy variable 1-8 OS = Homo sapiens GN = IGHV1-8 |
| | PE = 3 SV = 1 |
| P0DP23 | Calmodulin-1 OS = Homo sapiens GN = CALM1 PE = 1 SV = 1 |
| P10606 | Cytochrome c oxidase subunit 5B, mitochondrial OS = Homo sapiens |
| | GN = COX5B PE = 1 SV = 2 |
| P11166 | Solute carrier family 2, facilitated glucose transporter member 1 |
| | OS = Homo sapiens GN = SLC2A1 PE = 1 SV = 2 |
| P11169 | Solute carrier family 2, facilitated glucose transporter member 3 |
| | OS = Homo sapiens GN = SLC2A3 PE = 1 SV = 1 |
| P11215 | Integrin alpha-M OS = Homo sapiens GN = ITGAM PE = 1 SV = 2 |
| P11226 | Mannose-binding protein C OS = Homo sapiens GN = MBL2 PE = 1 |
| | SV = 2 |
| P11279 | Lysosome-associated membrane glycoprotein 1 OS = Homo sapiens |
| | GN = LAMP1 PE = 1 SV = 3 |
| P11532 | Dystrophin OS = Homo sapiens GN = DMD PE = 1 SV = 3 |
| P11678 | Eosinophil peroxidase OS = Homo sapiens GN = EPX PE = 1 SV = 2 |
| P12429 | Annexin A3 OS = Homo sapiens GN = ANXA3 PE = 1 SV = 3 |
| P13224 | Platelet glycoprotein Ib beta chain OS = Homo sapiens GN = GP1BB |
| | PE = 1 SV = 1 |
| P13667 | Protein disulfide-isomerase A4 OS = Homo sapiens GN = PDIA4 PE = 1 |
| | SV = 2 |
| P13671 | Complement component C6 OS = Homo sapiens GN = C6 PE = 1 SV = 3 |
| P13798 | Acylamino-acid-releasing enzyme OS = Homo sapiens GN = APEH |
| | PE = 1 SV = 4 |
| P15144 | Aminopeptidase N OS = Homo sapiens GN = ANPEP PE = 1 SV = 4 |
| P16035 | Metalloproteinase inhibitor 2 OS = Homo sapiens GN = TIMP2 PE = 1 |
| | SV = 2 |
| P16112 | Aggrecan core protein OS = Homo sapiens GN = ACAN PE = 1 SV = 2 |
| P16157 | Ankyrin-1 OS = Homo sapiens GN = ANK1 PE = 1 SV = 3 |
| P17301 | Integrin alpha-2 OS = Homo sapiens GN = ITGA2 PE = 1 SV = 1 |
| P17987 | T-complex protein 1 subunit alpha OS = Homo sapiens GN = TCP1 |
| | PE = 1 SV = 1 |
| P19652 | Alpha-1-acid glycoprotein 2 OS = Homo sapiens GN = ORM2 PE = 1 |
| | SV = 2 |
| P19823 | Inter-alpha-trypsin inhibitor heavy chain H2 OS = Homo sapiens |
| | GN = ITIH2 PE = 1 SV = 2 |
| P20851 | C4b-binding protein beta chain OS = Homo sapiens GN = C4BPB PE = 1 |
| | SV = 1 |
| P21980 | Protein-glutamine gamma-glutamyltransferase 2 OS = Homo sapiens |
| | GN = TGM2 PE = 1 SV = 2 |
| P22792 | Carboxypeptidase N subunit 2 OS = Homo sapiens GN = CPN2 PE = 1 |
| | SV = 3 |
| P23083 | Immunoglobulin heavy variable 1-2 OS = Homo sapiens GN = IGHV1-2 |
| | PE = 1 SV = 2 |
| P23526 | Adenosylhomocysteinase OS = Homo sapiens GN = AHCY PE = 1 SV = 4 |
| P23743 | Diacylglycerol kinase alpha OS = Homo sapiens GN = DGKA PE = 1 |
| | SV = 3 |
| P24043 | Laminin subunit alpha-2 OS = Homo sapiens GN = LAMA2 PE = 1 SV = 4 |
| P25311 | Zinc-alpha-2-glycoprotein OS = Homo sapiens GN = AZGP1 PE = 1 |
| | SV = 2 |
| P27105 | Erythrocyte band 7 integral membrane protein OS = Homo sapiens |
| | GN = STOM PE = 1 SV = 3 |
| P27169 | Serum paraoxonase/arylesterase 1 OS = Homo sapiens GN = PON1 |
| | PE = 1 SV = 3 |
| P27361 | Mitogen-activated protein kinase 3 OS = Homo sapiens GN = MAPK3 |
| | PE = 1 SV = 4 |
| P27816 | Microtubule-associated protein 4 OS = Homo sapiens GN = MAP4 PE = 1 |
| | SV = 3 |
| P28074 | Proteasome subunit beta type-5 OS = Homo sapiens GN = PSMB5 |
| | PE = 1 SV = 3 |
| P29622 | Kallistatin OS = Homo sapiens GN = SERPINA4 PE = 1 SV = 3 |
| P33981 | Dual specificity protein kinase TTK OS = Homo sapiens GN = TTK |
| | PE = 1 SV = 2 |
| P34897 | Serine hydroxymethyltransferase, mitochondrial OS = Homo sapiens |
| | GN = SHMT2 PE = 1 SV = 3 |
| P35443 | Thrombospondin-4 OS = Homo sapiens GN = THBS4 PE = 1 SV = 2 |
| P35542 | Serum amyloid A-4 protein OS = Homo sapiens GN = SAA4 PE = 1 SV = 2 |
| P35858 | Insulin-like growth factor-binding protein complex acid labile subunit |
| | OS = Homo sapiens GN = IGFALS PE = 1 SV = 1 |
| P36543 | V-type proton ATPase subunit E 1 OS = Homo sapiens |
| | GN = ATP6V1E1 PE = 1 SV = 1 |
| P36955 | Pigment epithelium-derived factor OS = Homo sapiens GN = SERPINF1 |
| | PE = 1 SV = 4 |
| P38405 | Guanine nucleotide-binding protein G(olf) subunit alpha OS = Homo |
| | sapiens GN = GNAL PE = 1 SV = 1 |
| P39059 | Collagen alpha-1(XV) chain OS = Homo sapiens GN = COL15A1 PE = 1 |
| | SV = 2 |
| P40123 | Adenylyl cyclase-associated protein 2 OS = Homo sapiens GN = CAP2 |
| | PE = 1 SV = 1 |
| P40227 | T-complex protein 1 subunit zeta OS = Homo sapiens GN = CCT6A |
| | PE = 1 SV = 3 |
| P42704 | Leucine-rich PPR motif-containing protein, mitochondrial OS = Homo |
| | sapiens GN = LRPPRC PE = 1 SV = 3 |
| P43243 | Matrin-3 OS = Homo sapiens GN = MATR3 PE = 1 SV = 2 |
| P43652 | Afamin OS = Homo sapiens GN = AFM PE = 1 SV = 1 |
| P46939 | Utrophin OS = Homo sapiens GN = UTRN PE = 1 SV = 2 |
| P46940 | Ras GTPase-activating-like protein IQGAP1 OS = Homo sapiens |
| | GN = IQGAP1 PE = 1 SV = 1 |
| P46977 | Dolichyl-diphosphooligosaccharide-protein glycosyltransferase |
| | subunit STT3A OS = Homo sapiens GN = STT3A PE = 1 SV = 2 |
| P48454 | Serine/threonine-protein phosphatase 2B catalytic subunit gamma |
| | isoform OS = Homo sapiens GN = PPP3CC PE = 1 SV = 3 |
| P49257 | Protein ERGIC-53 OS = Homo sapiens GN = LMAN1 PE = 1 SV = 2 |
| P49863 | Granzyme K OS = Homo sapiens GN = GZMK PE = 1 SV = 1 |
| P49908 | Selenoprotein P OS = Homo sapiens GN = SELENOP PE = 1 SV = 3 |
| P50749 | Ras association domain-containing protein 2 OS = Homo sapiens |
| | GN = RASSF2 PE = 1 SV = 1 |
| P52272 | Heterogeneous nuclear ribonucleoprotein M OS = Homo sapiens |
| | GN = HNRNPM PE = 1 SV = 3 |
| P52907 | F-actin-capping protein subunit alpha-1 OS = Homo sapiens |
| | GN = CAPZA1 PE = 1 SV = 3 |
| P53618 | Coatomer subunit beta OS = Homo sapiens GN = COPB1 PE = 1 SV = 3 |
| P53621 | Coatomer subunit alpha OS = Homo sapiens GN = COPA PE = 1 SV = 2 |
| P53999 | Activated RNA polymerase II transcriptional coactivator p15 |
| | OS = Homo sapiens GN = SUB1 PE = 1 SV = 3 |
| P55290 | Cadherin-13 OS = Homo sapiens GN = CDH13 PE = 1 SV = 1 |
| P61247 | 40S ribosomal protein S3a OS = Homo sapiens GN = RPS3A PE = 1 |
| | SV = 2 |
| P80748 | Immunoglobulin lambda variable 3-21 OS = Homo sapiens GN = IGLV3- |
| | 21 PE = 1 SV = 2 |
| Q00341 | Vigilin OS = Homo sapiens GN = HDLBP PE = 1 SV = 2 |
| Q01546 | Keratin, type II cytoskeletal 2 oral OS = Homo sapiens GN = KRT76 |
| | PE = 1 SV = 2 |
| Q02809 | Procollagen-lysine,2-oxoglutarate 5-dioxygenase 1 OS = Homo |
| | sapiens GN = PLOD1 PE = 1 SV = 2 |
| Q09666 | Neuroblast differentiation-associated protein AHNAK OS = Homo |
| | sapiens GN = AHNAK PE = 1 SV = 2 |
| Q10471 | Polypeptide N-acetylgalactosaminyltransferase 2 OS = Homo sapiens |
| | GN = GALNT2 PE = 1 SV = 1 |
| Q12931 | Heat shock protein 75 kDa, mitochondrial OS = Homo sapiens |
| | GN = TRAP1 PE = 1 SV = 3 |
| Q13554 | Calcium/calmodulin-dependent protein kinase type II subunit beta |
| | OS = Homo sapiens GN = CAMK2B PE = 1 SV = 3 |
| Q13790 | Apolipoprotein F OS = Homo sapiens GN = APOF PE = 1 SV = 2 |
| Q13813 | Spectrin alpha chain, non-erythrocytic 1 OS = Homo sapiens |
| | GN = SPTAN1 PE = 1 SV = 3 |
| Q13976 | cGMP-dependent protein kinase 1 OS = Homo sapiens GN = PRKG1 |
| | PE = 1 SV = 3 |
| Q14161 | ARF GTPase-activating protein GIT2 OS = Homo sapiens GN = GIT2 |
| | PE = 1 SV = 2 |
| Q14568 | Heat shock protein HSP 90-alpha A2 OS = Homo sapiens |
| | GN = HSP90AA2P PE = 1 SV = 2 |
| Q14624 | Inter-alpha-trypsin inhibitor heavy chain H4 OS = Homo sapiens |
| | GN = ITIH4 PE = 1 SV = 4 |
| Q14683 | Structural maintenance of chromosomes protein 1A OS = Homo |
| | sapiens GN = SMC1A PE = 1 SV = 2 |
| Q14BN4 | Sarcolemmal membrane-associated protein OS = Homo sapiens |
| | GN = SLMAP PE = 1 SV = 1 |
| Q15029 | 116 kDa U5 small nuclear ribonucleoprotein component OS = Homo |
| | sapiens GN = EFTUD2 PE = 1 SV = 1 |
| Q15436 | Protein transport protein Sec23A OS = Homo sapiens GN = SEC23A |
| | PE = 1 SV = 2 |
| Q15477 | Helicase SKI2W OS = Homo sapiens GN = SKIV2L PE = 1 SV = 3 |
| Q16610 | Extracellular matrix protein 1 OS = Homo sapiens GN = ECM1 PE = 1 |
| | SV = 2 |
| Q2M2I5 | Keratin, type I cytoskeletal 24 OS = Homo sapiens GN = KRT24 PE = 1 |
| | SV = 1 |
| Q53H12 | Acylglycerol kinase, mitochondrial OS = Homo sapiens GN = AGK PE = 1 |
| | SV = 2 |
| Q56UQ5 | TPT1-like protein OS = Homo sapiens PE = 2 SV = 2 |
| Q58FG0 | Putative heat shock protein HSP 90-alpha A5 OS = Homo sapiens |
| | GN = HSP90AA5P PE = 2 SV = 1 |
| Q5SSJ5 | Heterochromatin protein 1-binding protein 3 OS = Homo sapiens |
| | GN = HP1BP3 PE = 1 SV = 1 |
| Q5TDH0 | Protein DDI1 homolog 2 OS = Homo sapiens GN = DDI2 PE = 1 SV = 1 |
| Q6EEV6 | Small ubiquitin-related modifier 4 OS = Homo sapiens GN = SUMO4 |
| | PE = 1 SV = 2 |
| Q6P4A8 | Phospholipase B-like 1 OS = Homo sapiens GN = PLBD1 PE = 1 SV = 2 |
| Q6UX71 | Plexin domain-containing protein 2 OS = Homo sapiens GN = PLXDC2 |
| | PE = 1 SV = 1 |
| Q6ZU15 | Septin-14 OS = Homo sapiens GN = SEPT14 PE = 1 SV = 2 |
| Q7Z3Y8 | Keratin, type I cytoskeletal 27 OS = Homo sapiens GN = KRT27 PE = 1 |
| | SV = 2 |
| Q86W34 | Archaemetzincin-2 OS = Homo sapiens GN = AMZ2 PE = 1 SV = 2 |
| Q86Y07 | Serine/threonine-protein kinase VRK2 OS = Homo sapiens GN = VRK2 |
| | PE = 1 SV = 3 |
| Q8IZ83 | Aldehyde dehydrogenase family 16 member A1 OS = Homo sapiens |
| | GN = ALDH16A1 PE = 1 SV = 2 |
| Q8IZF3 | Adhesion G protein-coupled receptor F4 OS = Homo sapiens |
| | GN = ADGRF4 PE = 2 SV = 3 |
| Q8IZJ4 | Ral-GDS-related protein OS = Homo sapiens GN = RGL4 PE = 2 SV = 1 |
| Q8N568 | Serine/threonine-protein kinase DCLK2 OS = Homo sapiens |
| | GN = DCLK2 PE = 1 SV = 4 |
| Q8NFI4 | Putative protein FAM10A5 OS = Homo sapiens GN = ST13P5 PE = 5 |
| | SV = 1 |
| Q8TAD4 | Zinc transporter 5 OS = Homo sapiens GN = SLC30A5 PE = 1 SV = 1 |
| Q8WUJ3 | Cell migration-inducing and hyaluronan-binding protein OS = Homo |
| | sapiens GN = CEMIP PE = 1 SV = 2 |
| Q8WY36 | HMG box transcription factor BBX OS = Homo sapiens GN = BBX PE = 1 |
| | SV = 1 |
| Q92764 | Keratin, type I cuticular Ha5 OS = Homo sapiens GN = KRT35 PE = 1 |
| | SV = 5 |
| Q92804 | TATA-binding protein-associated factor 2N OS = Homo sapiens |
| | GN = TAF15 PE = 1 SV = 1 |
| Q92841 | Probable ATP-dependent RNA helicase DDX17 OS = Homo sapiens |
| | GN = DDX17 PE = 1 SV = 2 |
| Q96AE4 | Far upstream element-binding protein 1 OS = Homo sapiens |
| | GN = FUBP1 PE = 1 SV = 3 |
| Q96C19 | EF-hand domain-containing protein D2 OS = Homo sapiens |
| | GN = EFHD2 PE = 1 SV = 1 |
| Q96C23 | Aldose 1-epimerase OS = Homo sapiens GN = GALM PE = 1 SV = 1 |
| Q96CV9 | Optineurin OS = Homo sapiens GN = OPTN PE = 1 SV = 2 |
| Q96HC4 | PDZ and LIM domain protein 5 OS = Homo sapiens GN = PDLIM5 |
| | PE = 1 SV = 5 |
| Q96PD5 | N-acetylmuramoyl-L-alanine amidase OS = Homo sapiens |
| | GN = PGLYRP2 PE = 1 SV = 1 |
| Q96Q15 | Serine/threonine-protein kinase SMG1 OS = Homo sapiens GN = SMG1 |
| | PE = 1 SV = 3 |
| Q96RW7 | Hemicentin-1 OS = Homo sapiens GN = HMCN1 PE = 1 SV = 2 |
| Q99439 | Calponin-2 OS = Homo sapiens GN = CNN2 PE = 1 SV = 4 |
| Q99733 | Nucleosome assembly protein 1-like 4 OS = Homo sapiens |
| | GN = NAP1L4 PE = 1 SV = 1 |
| Q99816 | Tumor susceptibility gene 101 protein OS = Homo sapiens |
| | GN = TSG101 PE = 1 SV = 2 |
| Q99969 | Retinoic acid receptor responder protein 2 OS = Homo sapiens |
| | GN = RARRES2 PE = 1 SV = 1 |
| Q9BQL6 | Fermitin family homolog 1 OS = Homo sapiens GN = FERMT1 PE = 1 |
| | SV = 1 |
| Q9BTV4 | Transmembrane protein 43 OS = Homo sapiens GN = TMEM43 PE = 1 |
| | SV = 1 |
| Q9BXN1 | Asporin OS = Homo sapiens GN = ASPN PE = 1 SV = 2 |
| Q9BXR6 | Complement factor H-related protein 5 OS = Homo sapiens |
| | GN = CFHR5 PE = 1 SV = 1 |
| Q9BZQ8 | Protein Niban OS = Homo sapiens GN = FAM129A PE = 1 SV = 1 |
| Q9H0Q0 | Protein FAM49A OS = Homo sapiens GN = FAM49A PE = 2 SV = 1 |
| Q9H0R4 | Haloacid dehalogenase-like hydrolase domain-containing protein 2 |
| | OS = Homo sapiens GN = HDHD2 PE = 1 SV = 1 |
| Q9H115 | Beta-soluble NSF attachment protein OS = Homo sapiens GN = NAPB |
| | PE = 1 SV = 2 |
| Q9H6S0 | Probable ATP-dependent RNA helicase YTHDC2 OS = Homo sapiens |
| | GN = YTHDC2 PE = 1 SV = 2 |
| Q9HAV7 | GrpE protein homolog 1, mitochondrial OS = Homo sapiens |
| | GN = GRPEL1 PE = 1 SV = 2 |
| Q9NRP0 | Oligosaccharyltransferase complex subunit OSTC OS = Homo sapiens |
| | GN = OSTC PE = 1 SV = 1 |
| Q9NTJ5 | Phosphatidylinositide phosphatase SAC1 OS = Homo sapiens |
| | GN = SACM1L PE = 1 SV = 2 |
| Q9NYA4 | Myotubularin-related protein 4 OS = Homo sapiens GN = MTMR4 PE = 1 |
| | SV = 2 |
| Q9NZI8 | Insulin-like growth factor 2 mRNA-binding protein 1 OS = Homo |
| | sapiens GN = IGF2BP1 PE = 1 SV = 2 |
| Q9NZM1 | Myoferlin OS = Homo sapiens GN = MYOF PE = 1 SV = 1 |
| Q9P2D1 | Chromodomain-helicase-DNA-binding protein 7 OS = Homo sapiens |
| | GN = CHD7 PE = 1 SV = 3 |
| Q9P2E9 | Ribosome-binding protein 1 OS = Homo sapiens GN = RRBP1 PE = 1 |
| | SV = 4 |
| Q9UBG0 | C-type mannose receptor 2 OS = Homo sapiens GN = MRC2 PE = 1 |
| | SV = 2 |
| Q9UGI8 | Testin OS = Homo sapiens GN = TES PE = 1 SV = 1 |
| Q9UI15 | Transgelin-3 OS = Homo sapiens GN = TAGLN3 PE = 1 SV = 2 |
| Q9UJZ1 | Stomatin-like protein 2, mitochondrial OS = Homo sapiens |
| | GN = STOML2 PE = 1 SV = 1 |
| Q9UMX0 | Ubiquilin-1 OS = Homo sapiens GN = UBQLN1 PE = 1 SV = 2 |
| Q9UNM6 | 26S proteasome non-ATPase regulatory subunit 13 OS = Homo |
| | sapiens GN = PSMD13 PE = 1 SV = 2 |
| Q9UQ03 | Coronin-2B OS = Homo sapiens GN = CORO2B PE = 1 SV = 4 |
| Q9Y2J0 | Rabphilin-3A OS = Homo sapiens GN = RPH3A PE = 1 SV = 1 |
| Q9Y490 | Talin-1 OS = Homo sapiens GN = TLN1 PE = 1 SV = 3 |
| Q9Y625 | Glypican-6 OS = Homo sapiens GN = GPC6 PE = 1 SV = 1 |
| Q9Y639 | Neuroplastin OS = Homo sapiens GN = NPTN PE = 1 SV = 2 |

Experiment 4: Comparison Between Thrombus Culture Supernatant Vs Controls

It was observed that 125 proteins were significantly more abundant in the supernatant obtained from culture of the thrombus tissue compared with the control samples. These proteins are listed in Table 4 below and referred to in the following discussion as Group D proteins or ‘Thrombus supernatant’.

TABLE 4
Group D
Thrombus Supernatant

| UNIPROT ID | Description |
|---|---|
| A0A075B6K4 | Immunoglobulin lambda variable 3-10 OS = Homo sapiens GN = IGLV3- |
| | 10 PE = 3 SV = 2 |
| A0A075B6P5 | Immunoglobulin kappa variable 2-28 OS = Homo sapiens GN = IGKV2- |
| | 28 PE = 3 SV = 1 |
| A0A0B4J1U3 | Immunoglobulin lambda variable 1-36 OS = Homo sapiens GN = IGLV1- |
| | 36 PE = 3 SV = 5 |
| A0A0C4DH38 | Immunoglobulin heavy variable 5-51 OS = Homo sapiens GN = IGHV5- |
| | 51 PE = 3 SV = 1 |
| O00233 | 26S proteasome non-ATPase regulatory subunit 9 OS = Homo sapiens |
| | GN = PSMD9 PE = 1 SV = 3 |
| O00505 | Importin subunit alpha-4 OS = Homo sapiens GN = KPNA3 PE = 1 SV = 2 |
| O14787 | Transportin-2 OS = Homo sapiens GN = TNPO2 PE = 1 SV = 3 |
| O43865 | S-adenosylhomocysteine hydrolase-like protein 1 OS = Homo sapiens |
| | GN = AHCYL1 PE = 1 SV = 2 |
| O60741 | Potassium/sodium hyperpolarization-activated cyclic nucleotide-gated |
| | channel 1 OS = Homo sapiens GN = HCN1 PE = 1 SV = 3 |
| O94788 | Retinal dehydrogenase 2 OS = Homo sapiens GN = ALDH1A2 PE = 1 |
| | SV = 3 |
| P00734 | Prothrombin OS = Homo sapiens GN = F2 PE = 1 SV = 2 |
| P00739 | Haptoglobin-related protein OS = Homo sapiens GN = HPR PE = 2 SV = 2 |
| P00747 | Plasminogen OS = Homo sapiens GN = PLG PE = 1 SV = 2 |
| P00915 | Carbonic anhydrase 1 OS = Homo sapiens GN = CA1 PE = 1 SV = 2 |
| P01019 | Angiotensinogen OS = Homo sapiens GN = AGT PE = 1 SV = 1 |
| P01024 | Complement C3 OS = Homo sapiens GN = C3 PE = 1 SV = 2 |
| P01042 | Kininogen-1 OS = Homo sapiens GN = KNG1 PE = 1 SV = 2 |
| P01817 | Immunoglobulin heavy variable 2-5 OS = Homo sapiens GN = IGHV2-5 |
| | PE = 1 SV = 2 |
| P01824 | Immunoglobulin heavy variable 4-39 OS = Homo sapiens GN = IGHV4- |
| | 39 PE = 1 SV = 2 |
| P01834 | Immunoglobulin kappa constant OS = Homo sapiens GN = IGKC PE = 1 |
| | SV = 2 |
| P01859 | Immunoglobulin heavy constant gamma 2 OS = Homo sapiens |
| | GN = IGHG2 PE = 1 SV = 2 |
| P01861 | Immunoglobulin heavy constant gamma 4 OS = Homo sapiens |
| | GN = IGHG4 PE = 1 SV = 1 |
| P02042 | Hemoglobin subunit delta OS = Homo sapiens GN = HBD PE = 1 SV = 2 |
| P02675 | Fibrinogen beta chain OS = Homo sapiens GN = FGB PE = 1 SV = 2 |
| P02679 | Fibrinogen gamma chain OS = Homo sapiens GN = FGG PE = 1 SV = 3 |
| P02760 | Protein AMBP OS = Homo sapiens GN = AMBP PE = 1 SV = 1 |
| P02763 | Alpha-1-acid glycoprotein 1 OS = Homo sapiens GN = ORM1 PE = 1 |
| | SV = 1 |
| P02766 | Transthyretin OS = Homo sapiens GN = TTR PE = 1 SV = 1 |
| P02787 | Serotransferrin OS = Homo sapiens GN = TF PE = 1 SV = 3 |
| P02790 | Hemopexin OS = Homo sapiens GN = HPX PE = 1 SV = 2 |
| P02794 | Ferritin heavy chain OS = Homo sapiens GN = FTH1 PE = 1 SV = 2 |
| P03952 | Plasma kallikrein OS = Homo sapiens GN = KLKB1 PE = 1 SV = 1 |
| P04003 | C4b-binding protein alpha chain OS = Homo sapiens GN = C4BPA |
| | PE = 1 SV = 2 |
| P04004 | Vitronectin OS = Homo sapiens GN = VTN PE = 1 SV = 1 |
| P04196 | Histidine-rich glycoprotein OS = Homo sapiens GN = HRG PE = 1 SV = 1 |
| P04217 | Alpha-1 B-glycoprotein OS = Homo sapiens GN = A1BG PE = 1 SV = 4 |
| P05121 | Plasminogen activator inhibitor 1 OS = Homo sapiens GN = SERPINE1 |
| | PE = 1 SV = 1 |
| P05156 | Complement factor I OS = Homo sapiens GN = CFI PE = 1 SV = 2 |
| P05160 | Coagulation factor XIII B chain OS = Homo sapiens GN = F13B PE = 1 |
| | SV = 3 |
| P06727 | Apolipoprotein A-IV OS = Homo sapiens GN = APOA4 PE = 1 SV = 3 |
| P07358 | Complement component C8 beta chain OS = Homo sapiens GN = C8B |
| | PE = 1 SV = 3 |
| P07360 | Complement component C8 gamma chain OS = Homo sapiens |
| | GN = C8G PE = 1 SV = 3 |
| P07954 | Fumarate hydratase, mitochondrial OS = Homo sapiens GN = FH PE = 1 |
| | SV = 3 |
| P08246 | Neutrophil elastase OS = Homo sapiens GN = ELANE PE = 1 SV = 1 |
| P08697 | Alpha-2-antiplasmin OS = Homo sapiens GN = SERPINF2 PE = 1 SV = 3 |
| P0C0L4 | Complement C4-A OS = Homo sapiens GN = C4A PE = 1 SV = 2 |
| P12268 | Inosine-5′-monophosphate dehydrogenase 2 OS = Homo sapiens |
| | GN = IMPDH2 PE = 1 SV = 2 |
| P13611 | Versican core protein OS = Homo sapiens GN = VCAN PE = 1 SV = 3 |
| P14314 | Glucosidase 2 subunit beta OS = Homo sapiens GN = PRKCSH PE = 1 |
| | SV = 2 |
| P17066 | Heat shock 70 kDa protein 6 OS = Homo sapiens GN = HSPA6 PE = 1 |
| | SV = 2 |
| P18428 | Lipopolysaccharide-binding protein OS = Homo sapiens GN = LBP |
| | PE = 1 SV = 3 |
| P19367 | Hexokinase-1 OS = Homo sapiens GN = HK1 PE = 1 SV = 3 |
| P19652 | Alpha-1-acid glycoprotein 2 OS = Homo sapiens GN = ORM2 PE = 1 |
| | SV = 2 |
| P19827 | Inter-alpha-trypsin inhibitor heavy chain H1 OS = Homo sapiens |
| | GN = ITIH1 PE = 1 SV = 3 |
| P20851 | C4b-binding protein beta chain OS = Homo sapiens GN = C4BPB PE = 1 |
| | SV = 1 |
| P21281 | V-type proton ATPase subunit B, brain isoform OS = Homo sapiens |
| | GN = ATP6V1B2 PE = 1 SV = 3 |
| P23470 | Receptor-type tyrosine-protein phosphatase gamma OS = Homo |
| | sapiens GN = PTPRG PE = 1 SV = 4 |
| P25705 | ATP synthase subunit alpha, mitochondrial OS = Homo sapiens |
| | GN = ATP5A1 PE = 1 SV = 1 |
| P25940 | Collagen alpha-3(V) chain OS = Homo sapiens GN = COL5A3 PE = 1 |
| | SV = 3 |
| P26368 | Splicing factor U2AF 65 kDa subunit OS = Homo sapiens GN = U2AF2 |
| | PE = 1 SV = 4 |
| P27169 | Serum paraoxonase/arylesterase 1 OS = Homo sapiens GN = PON1 |
| | PE = 1 SV = 3 |
| P28062 | Proteasome subunit beta type-8 OS = Homo sapiens GN = PSMB8 |
| | PE = 1 SV = 3 |
| P28698 | Myeloid zinc finger 1 OS = Homo sapiens GN = MZF1 PE = 1 SV = 3 |
| P29590 | Protein PML OS = Homo sapiens GN = PML PE = 1 SV = 3 |
| P29622 | Kallistatin OS = Homo sapiens GN = SERPINA4 PE = 1 SV = 3 |
| P33176 | Kinesin-1 heavy chain OS = Homo sapiens GN = KIF5B PE = 1 SV = 1 |
| P35442 | Thrombospondin-2 OS = Homo sapiens GN = THBS2 PE = 1 SV = 2 |
| P35612 | Beta-adducin OS = Homo sapiens GN = ADD2 PE = 1 SV = 3 |
| P35637 | RNA-binding protein FUS OS = Homo sapiens GN = FUS PE = 1 SV = 1 |
| P42858 | Huntingtin OS = Homo sapiens GN = HTT PE = 1 SV = 2 |
| P43652 | Afamin OS = Homo sapiens GN = AFM PE = 1 SV = 1 |
| P45974 | Ubiquitin carboxyl-terminal hydrolase 5 OS = Homo sapiens GN = USP5 |
| | PE = 1 SV = 2 |
| P46777 | 60S ribosomal protein L5 OS = Homo sapiens GN = RPL5 PE = 1 SV = 3 |
| P46821 | Microtubule-associated protein 1B OS = Homo sapiens GN = MAP1B |
| | PE = 1 SV = 2 |
| P46939 | Utrophin OS = Homo sapiens GN = UTRN PE = 1 SV = 2 |
| P49908 | Selenoprotein P OS = Homo sapiens GN = SELENOP PE = 1 SV = 3 |
| P52789 | Hexokinase-2 OS = Homo sapiens GN = HK2 PE = 1 SV = 2 |
| P52790 | Hexokinase-3 OS = Homo sapiens GN = HK3 PE = 1 SV = 2 |
| P53675 | Clathrin heavy chain 2 OS = Homo sapiens GN = CLTCL1 PE = 1 SV = 2 |
| P55290 | Cadherin-13 OS = Homo sapiens GN = CDH13 PE = 1 SV = 1 |
| P59665 | Neutrophil defensin 1 OS = Homo sapiens GN = DEFA1 PE = 1 SV = 1 |
| P61201 | COP9 signalosome complex subunit 2 OS = Homo sapiens |
| | GN = COPS2 PE = 1 SV = 1 |
| P68400 | Casein kinase II subunit alpha OS = Homo sapiens GN = CSNK2A1 |
| | PE = 1 SV = 1 |
| P68871 | Hemoglobin subunit beta OS = Homo sapiens GN = HBB PE = 1 SV = 2 |
| P69905 | Hemoglobin subunit alpha OS = Homo sapiens GN = HBA1 PE = 1 SV = 2 |
| P78386 | Keratin, type II cuticular Hb5 OS = Homo sapiens GN = KRT85 PE = 1 |
| | SV = 1 |
| P80188 | Neutrophil gelatinase-associated lipocalin OS = Homo sapiens |
| | GN = LCN2 PE = 1 SV = 2 |
| P80748 | Immunoglobulin lambda variable 3-21 OS = Homo sapiens GN = IGLV3- |
| | 21 PE = 1 SV = 2 |
| Q03164 | Histone-Iysine N-methyltransferase 2A OS = Homo sapiens |
| | GN = KMT2A PE = 1 SV = 5 |
| Q05315 | Galectin-10 OS = Homo sapiens GN = CLC PE = 1 SV = 3 |
| Q07065 | Cytoskeleton-associated protein 4 OS = Homo sapiens GN = CKAP4 |
| | PE = 1 SV = 2 |
| Q14157 | Ubiquitin-associated protein 2-like OS = Homo sapiens GN = UBAP2L |
| | PE = 1 SV = 2 |
| Q14624 | Inter-alpha-trypsin inhibitor heavy chain H4 OS = Homo sapiens |
| | GN = ITIH4 PE = 1 SV = 4 |
| Q16610 | Extracellular matrix protein 1 OS = Homo sapiens GN = ECM1 PE = 1 |
| | SV = 2 |
| Q16881 | Thioredoxin reductase 1, cytoplasmic OS = Homo sapiens |
| | GN = TXNRD1 PE = 1 SV = 3 |
| Q58FG0 | Putative heat shock protein HSP 90-alpha A5 OS = Homo sapiens |
| | GN = HSP90AA5P PE = 2 SV = 1 |
| Q5VTE0 | Putative elongation factor 1-alpha-like 3 OS = Homo sapiens |
| | GN = EEF1A1P5 PE = 5 SV = 1 |
| Q7Z3Y7 | Keratin, type I cytoskeletal 28 OS = Homo sapiens GN = KRT28 PE = 1 |
| | SV = 2 |
| Q86VP6 | Cullin-associated NEDD8-dissociated protein 1 OS = Homo sapiens |
| | GN = CAND1 PE = 1 SV = 2 |
| Q8IUN9 | C-type lectin domain family 10 member A OS = Homo sapiens |
| | GN = CLEC10A PE = 1 SV = 1 |
| Q8NE71 | ATP-binding cassette sub-family F member 1 OS = Homo sapiens |
| | GN = ABCF1 PE = 1 SV = 2 |
| Q8NFY4 | Semaphorin-6D OS = Homo sapiens GN = SEMA6D PE = 1 SV = 1 |
| Q8TDB8 | Solute carrier family 2, facilitated glucose transporter member 14 |
| | OS = Homo sapiens GN = SLC2A14 PE = 2 SV = 1 |
| Q92747 | Actin-related protein 2/3 complex subunit 1A OS = Homo sapiens |
| | GN = ARPC1A PE = 2 SV = 2 |
| Q93008 | Probable ubiquitin carboxyl-terminal hydrolase FAF-X OS = Homo |
| | sapiens GN = USP9X PE = 1 SV = 3 |
| Q96AE4 | Far upstream element-binding protein 1 OS = Homo sapiens |
| | GN = FUBP1 PE = 1 SV = 3 |
| Q96BD5 | PHD finger protein 21A OS = Homo sapiens GN = PHF21A PE = 1 SV = 1 |
| Q96CW1 | AP-2 complex subunit mu OS = Homo sapiens GN = AP2M1 PE = 1 |
| | SV = 2 |
| Q96IY4 | Carboxypeptidase B2 OS = Homo sapiens GN = CPB2 PE = 1 SV = 2 |
| Q96PD5 | N-acetylmuramoyl-L-alanine amidase OS = Homo sapiens |
| | GN = PGLYRP2 PE = 1 SV = 1 |
| Q99439 | Calponin-2 OS = Homo sapiens GN = CNN2 PE = 1 SV = 4 |
| Q99490 | Arf-GAP with GTPase, ANK repeat and PH domain-containing protein |
| | 2 OS = Homo sapiens GN = AGAP2 PE = 1 SV = 2 |
| Q9BUF5 | Tubulin beta-6 chain OS = Homo sapiens GN = TUBB6 PE = 1 SV = 1 |
| Q9GZZ9 | Ubiquitin-like modifier-activating enzyme 5 OS = Homo sapiens |
| | GN = UBA5 PE = 1 SV = 1 |
| Q9H2M9 | Rab3 GTPase-activating protein non-catalytic subunit OS = Homo |
| | sapiens GN = RAB3GAP2 PE = 1 SV = 1 |
| Q9H2Y7 | Zinc finger protein 106 OS = Homo sapiens GN = ZNF106 PE = 1 SV = 1 |
| Q9HA64 | Ketosamine-3-kinase OS = Homo sapiens GN = FN3KRP PE = 1 SV = 2 |
| Q9NTJ4 | Alpha-mannosidase 2C1 OS = Homo sapiens GN = MAN2C1 PE = 1 |
| | SV = 1 |
| Q9NXG0 | Centlein OS = Homo sapiens GN = CNTLN PE = 1 SV = 5 |
| Q9P253 | Vacuolar protein sorting-associated protein 18 homolog OS = Homo |
| | sapiens GN = VPS18 PE = 1 SV = 2 |
| Q9P2R3 | Rabankyrin-5 OS = Homo sapiens GN = ANKFY1 PE = 1 SV = 2 |
| Q9UD71 | Protein phosphatase 1 regulatory subunit 1B OS = Homo sapiens |
| | GN = PPP1R1B PE = 1 SV = 2 |
| Q9Y4L1 | Hypoxia up-regulated protein 1 OS = Homo sapiens GN = HYOU1 PE = 1 |
| | SV = 1 |
| Q9Y5P4 | Collagen type IV alpha-3-binding protein OS = Homo sapiens |
| | GN = COL4A3BP PE = 1 SV = 1 |
| Q9Y6G9 | Cytoplasmic dynein 1 light intermediate chain 1 OS = Homo sapiens |
| | GN = DYNC1LI1 PE = 1 SV = 3 |

### Discussion

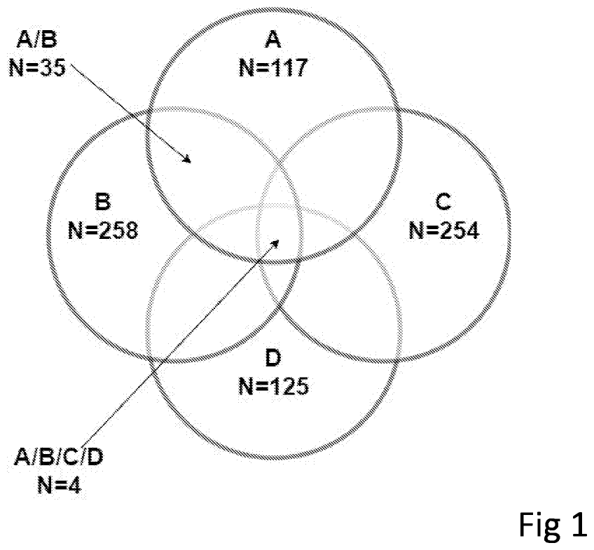

The results of the above experiments are represented in FIG. 1. Comparison of blood samples between patients with the fastest vs the slowest (n=10 each) showed 117 proteins to be differentially expressed in their plasma as Group A proteins, illustrated in FIG. 1. Group B comprised 258 proteins, of which 35 were also members of Group A, suggesting their origin to be from the AAA complex rather than any other physiological source. These proteins are listed in Table 5 below. Group C comprised 254 proteins and Group D comprised 125 proteins, as discussed above.

TABLE 5
Groups A & B intersection

| UNIPROT ID | Description |
|---|---|
| A0A0C4DH31 | Immunoglobulin heavy variable 1-18 OS = Homo sapiens GN = |
| | IGHV1-18 PE = 3 SV = 1 |
| P46821 | Microtubule-associated protein 1B OS = Homo sapiens GN = MAP1B |
| | PE = 1 SV = 2 |
| Q9NQH7 | Probable Xaa-Pro aminopeptidase 3 OS = Homo sapiens |
| | GN = XPNPEP3 PE = 1 SV = 1 |
| P08567 | Pleckstrin OS = Homo sapiens GN = PLEK PE = 1 SV = 3 |
| P00915 | Carbonic anhydrase 1 OS = Homo sapiens GN = CA1 PE = 1 SV = 2 |
| Q9H361 | Polyadenylate-binding protein 3 OS = Homo sapiens GN = PABPC3 |
| | PE = 1 SV = 2 |
| Q96TC7 | Regulator of microtubule dynamics protein 3 OS = Homo sapiens |
| | GN = RMDN3 PE = 1 SV = 2 |
| P10643 | Complement component C7 OS = Homo sapiens GN = C7 PE = 1 SV = 2 |
| P06727 | Apolipoprotein A-IV OS = Homo sapiens GN = APOA4 PE = 1 SV = 3 |
| O00555 | Voltage-dependent P/Q-type calcium channel subunit alpha-1A |
| | OS = Homo sapiens GN = CACNA1A PE = 1 SV = 2 |
| Q96SN8 | CDK5 regulatory subunit-associated protein 2 OS = Homo sapiens |
| | GN = CDK5RAP2 PE = 1 SV = 5 |
| Q8WZ75 | Roundabout homolog 4 OS = Homo sapiens GN = ROBO4 PE = 1 SV = 1 |
| P27797 | Calreticulin OS = Homo sapiens GN = CALR PE = 1 SV = 1 |
| Q14204 | Cytoplasmic dynein 1 heavy chain 1 OS = Homo sapiens |
| | GN = DYNC1H1 PE = 1 SV = 5 |
| Q58FG0 | Putative heat shock protein HSP 90-alpha A5 OS = Homo sapiens |
| | GN = HSP90AA5P PE = 2 SV = 1 |
| P07099 | Epoxide hydrolase 1 OS = Homo sapiens GN = EPHX1 PE = 1 SV = 1 |
| P21810 | Biglycan OS = Homo sapiens GN = BGN PE = 1 SV = 2 |
| P57721 | Poly(rC)-binding protein 3 OS = Homo sapiens GN = PCBP3 PE = 2 |
| | SV = 2 |
| P61916 | Epididymal secretory protein E1 OS = Homo sapiens GN = NPC2 PE = 1 |
| | SV = 1 |
| Q13093 | Platelet-activating factor acetylhydrolase OS = Homo sapiens |
| | GN = PLA2G7 PE = 1 SV = 1 |
| P08729 | Keratin, type II cytoskeletal 7 OS = Homo sapiens GN = KRT7 PE = 1 |
| | SV = 5 |
| O95568 | Histidine protein methyltransferase 1 homolog OS = Homo sapiens |
| | GN = METTL18 PE = 1 SV = 1 |
| Q12906 | Interleukin enhancer-binding factor 3 OS = Homo sapiens GN = ILF3 |
| | PE = 1 SV = 3 |
| O75882 | Attractin OS = Homo sapiens GN = ATRN PE = 1 SV = 2 |
| P31948 | Stress-induced-phosphoprotein 1 OS = Homo sapiens GN = STIP1 |
| | PE = 1 SV = 1 |
| Q9BZA8 | Protocadherin-11 Y-linked OS = Homo sapiens GN = PCDH11Y PE = 1 |
| | SV = 1 |
| P10809 | 60 kDa heat shock protein, mitochondrial OS = Homo sapiens |
| | GN = HSPD1 PE = 1 SV = 2 |
| Q4VNC0 | Probable cation-transporting ATPase 13A5 OS = Homo sapiens |
| | GN = ATP13A5 PE = 2 SV = 1 |
| P01764 | Immunoglobulin heavy variable 3-23 OS = Homo sapiens GN = IGHV3- |
| | 23 PE = 1 SV = 2 |
| P07360 | Complement component C8 gamma chain OS = Homo sapiens |
| | GN = C8G PE = 1 SV = 3 |
| Q99497 | Protein/nucleic acid deglycase DJ-1 OS = Homo sapiens GN = PARK7 |
| | PE = 1 SV = 2 |
| P51884 | Lumican OS = Homo sapiens GN = LUM PE = 1 SV = 2 |
| Q15847 | Adipogenesis regulatory factor OS = Homo sapiens GN = ADIRF PE = 1 |
| | SV = 1 |
| P81172 | Hepcidin OS = Homo sapiens OX = 9606 GN = HAMP PE = 1 SV = 2 |

Comparison of the proteomics profile of aneurysm tissue, ILT, and omental artery show 128 proteins to be uniquely present in ILT. Analyses of the tissue culture supernatant further revealed four proteins: (i) that are uniquely present in ILT; (ii) that are released by ILT; (iii) systemic levels of which change after AAA surgery; and (iv) which differ between fast and slow growth AAAs.

These proteins are attractin (UniProt ID 075882), Apolipoprotein A4 (UniProt ID P06727), Complement C8 (UniProt ID P07360) and HSP90AA5P (UniProt ID Q58FG0).

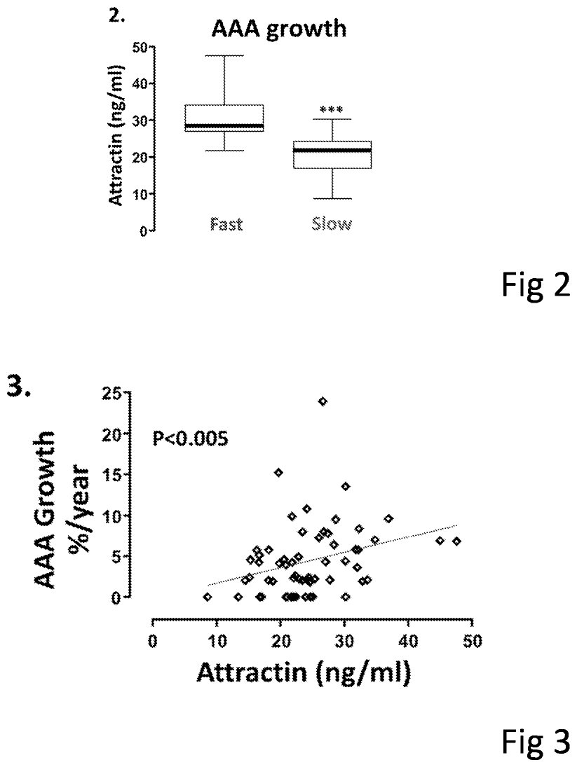

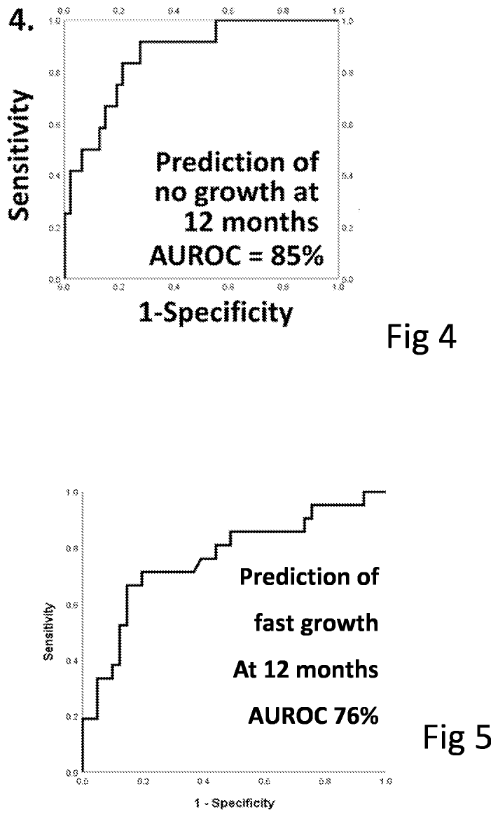

To validate the LC-MSMS data, attractin was selected for further study. Attractin is present in Groups A, B and C. The attractin level in the bloodstream of an individual patient was measured by ELISA (R&D Quantikine DATRNO). Plasma attractin level is significantly higher in patients with fast AAA growth (FIG. 2, median 28.5 vs 21.9 ng/ml, P<0.001). Plasma attractin level correlates significantly with future AAA growth rate (FIG. 3, Spearman r=0.35, P<0.005). We regressed the measured values of attractin in combination with AAA diameter against a categorical response with levels of ‘Slow/No’ growth (0%) or growth (>0% growth), or fast growth (defined as more than the upper tertile of growth in this cohort) for outcomes at 12 months. Using attractin level and AAA diameter as input variables, the AUROC (Area Under the Receiver Operating Characteristics) for predicting slow/no growth of AAA at 12 months is 85% (FIG. 4, asymptotic P<0.001) and the AUROC for predicting fast growth of AAA at 12 months is 76% (FIG. 5 asymptomic P<0.005).

The data is set out in Table 6 below for slow or no growth over 12 months and Table 7 for fast growth.

TABLE 6
Slow/no growth
Common Rates and Indices for each Cut Off Value
Condition Variable No growth at 12 months
Estimated Prevalence = 0.21

| | Table Counts | | | | | | | | |
|---|---|---|---|---|---|---|---|---|---|
| Cutoff | TPs | FPs | FNs | TNs | TPR | TNR | | | TPR + |
| value | A | B | C | D | (Sens.) | (Spec.) | PPV | Accuracy | TNR |
| ≥0.00 | 13 | 49 | 0 | 0 | 1 | 0.0000 | 0.2097 | 0.2097 | 1.0000 |
| ≥0.01 | 13 | 48 | 0 | 1 | 1 | 0.0204 | 0.2131 | 0.2258 | 1.0204 |
| ≥0.01 | 13 | 47 | 0 | 2 | 1 | 0.0408 | 0.2167 | 0.2419 | 1.0408 |
| ≥0.02 | 13 | 46 | 0 | 3 | 1 | 0.0612 | 0.2203 | 0.2581 | 1.0612 |
| ≥0.02 | 13 | 45 | 0 | 4 | 1 | 0.0816 | 0.2241 | 0.2742 | 1.0816 |
| ≥0.02 | 13 | 44 | 0 | 5 | 1 | 0.1020 | 0.2281 | 0.2903 | 1.1020 |
| ≥0.03 | 13 | 43 | 0 | 6 | 1 | 0.1224 | 0.2321 | 0.3065 | 1.1224 |
| ≥0.03 | 13 | 42 | 0 | 7 | 1 | 0.1429 | 0.2364 | 0.3226 | 1.1429 |
| ≥0.03 | 13 | 41 | 0 | 8 | 1 | 0.1633 | 0.2407 | 0.3387 | 1.1633 |
| ≥0.03 | 13 | 40 | 0 | 9 | 1 | 0.1837 | 0.2453 | 0.3548 | 1.1837 |
| ≥0.03 | 13 | 39 | 0 | 10 | 1 | 0.2041 | 0.2500 | 0.3710 | 1.2041 |
| ≥0.04 | 13 | 38 | 0 | 11 | 1 | 0.2245 | 0.2549 | 0.3871 | 1.2245 |
| ≥0.04 | 13 | 37 | 0 | 12 | 1 | 0.2449 | 0.2600 | 0.4032 | 1.2449 |
| ≥0.04 | 13 | 36 | 0 | 13 | 1 | 0.2653 | 0.2653 | 0.4194 | 1.2653 |
| ≥0.04 | 13 | 35 | 0 | 14 | 1 | 0.2857 | 0.2708 | 0.4355 | 1.2857 |
| ≥0.05 | 13 | 33 | 0 | 16 | 1 | 0.3265 | 0.2826 | 0.4677 | 1.3265 |
| ≥0.05 | 13 | 32 | 0 | 17 | 1 | 0.3469 | 0.2889 | 0.4839 | 1.3469 |
| ≥0.05 | 13 | 31 | 0 | 18 | 1 | 0.3673 | 0.2955 | 0.5000 | 1.3673 |
| ≥0.06 | 13 | 30 | 0 | 19 | 1 | 0.3878 | 0.3023 | 0.5161 | 1.3878 |
| ≥0.07 | 13 | 29 | 0 | 20 | 1 | 0.4082 | 0.3095 | 0.5323 | 1.4082 |
| ≥0.07 | 12 | 29 | 1 | 20 | 0.9231 | 0.4082 | 0.2927 | 0.5161 | 1.3312 |
| ≥0.07 | 12 | 28 | 1 | 21 | 0.9231 | 0.4286 | 0.3000 | 0.5323 | 1.3516 |
| ≥0.08 | 12 | 27 | 1 | 22 | 0.9231 | 0.4490 | 0.3077 | 0.5484 | 1.3721 |
| ≥0.08 | 12 | 26 | 1 | 23 | 0.9231 | 0.4694 | 0.3158 | 0.5645 | 1.3925 |
| ≥0.08 | 12 | 25 | 1 | 24 | 0.9231 | 0.4898 | 0.3243 | 0.5806 | 1.4129 |
| ≥0.08 | 12 | 24 | 1 | 25 | 0.9231 | 0.5102 | 0.3333 | 0.5968 | 1.4333 |
| ≥0.10 | 12 | 23 | 1 | 26 | 0.9231 | 0.5306 | 0.3429 | 0.6129 | 1.4537 |
| ≥0.10 | 12 | 22 | 1 | 27 | 0.9231 | 0.5510 | 0.3529 | 0.6290 | 1.4741 |
| ≥0.11 | 12 | 21 | 1 | 28 | 0.9231 | 0.5714 | 0.3636 | 0.6452 | 1.4945 |
| ≥0.11 | 12 | 20 | 1 | 29 | 0.9231 | 0.5918 | 0.3750 | 0.6613 | 1.5149 |
| ≥0.12 | 12 | 19 | 1 | 30 | 0.9231 | 0.6122 | 0.3871 | 0.6774 | 1.5353 |
| ≥0.12 | 12 | 18 | 1 | 31 | 0.9231 | 0.6327 | 0.4000 | 0.6935 | 1.5557 |
| ≥0.15 | 12 | 17 | 1 | 32 | 0.9231 | 0.6531 | 0.4138 | 0.7097 | 1.5761 |
| ≥0.15 | 12 | 16 | 1 | 33 | 0.9231 | 0.6735 | 0.4286 | 0.7258 | 1.5965 |
| ≥0.17 | 12 | 15 | 1 | 34 | 0.9231 | 0.6939 | 0.4444 | 0.7419 | 1.6170 |
| ≥0.19 | 11 | 15 | 2 | 34 | 0.8462 | 0.6939 | 0.4231 | 0.7258 | 1.5400 |
| ≥0.20 | 11 | 14 | 2 | 35 | 0.8462 | 0.7143 | 0.4400 | 0.7419 | 1.5604 |
| ≥0.20 | 11 | 13 | 2 | 36 | 0.8462 | 0.7347 | 0.4583 | 0.7581 | 1.5808 |
| ≥0.22 | 10 | 13 | 3 | 36 | 0.7692 | 0.7347 | 0.4348 | 0.7419 | 1.5039 |
| ≥0.23 | 10 | 12 | 3 | 37 | 0.7692 | 0.7551 | 0.4545 | 0.7581 | 1.5243 |
| ≥0.24 | 9 | 12 | 4 | 37 | 0.6923 | 0.7551 | 0.4286 | 0.7419 | 1.4474 |
| ≥0.27 | 9 | 11 | 4 | 38 | 0.6923 | 0.7755 | 0.4500 | 0.7581 | 1.4678 |
| ≥0.28 | 8 | 11 | 5 | 38 | 0.6154 | 0.7755 | 0.4211 | 0.7419 | 1.3909 |
| ≥0.32 | 8 | 10 | 5 | 39 | 0.6154 | 0.7959 | 0.4444 | 0.7581 | 1.4113 |
| ≥0.33 | 8 | 9 | 5 | 40 | 0.6154 | 0.8163 | 0.4706 | 0.7742 | 1.4317 |
| ≥0.36 | 8 | 8 | 5 | 41 | 0.6154 | 0.8367 | 0.5000 | 0.7903 | 1.4521 |
| ≥0.36 | 8 | 7 | 5 | 42 | 0.6154 | 0.8571 | 0.5333 | 0.8065 | 1.4725 |
| ≥0.37 | 8 | 6 | 5 | 43 | 0.6154 | 0.8776 | 0.5714 | 0.8226 | 1.4929 |
| ≥0.37 | 8 | 5 | 5 | 44 | 0.6154 | 0.8980 | 0.6154 | 0.8387 | 1.5133 |
| ≥0.37 | 7 | 5 | 6 | 44 | 0.5385 | 0.8980 | 0.5833 | 0.8226 | 1.4364 |
| ≥0.39 | 7 | 4 | 6 | 45 | 0.5385 | 0.9184 | 0.6364 | 0.8387 | 1.4568 |
| ≥0.39 | 7 | 3 | 6 | 46 | 0.5385 | 0.9388 | 0.7000 | 0.8548 | 1.4772 |
| ≥0.42 | 6 | 3 | 7 | 46 | 0.4615 | 0.9388 | 0.6667 | 0.8387 | 1.4003 |
| ≥0.42 | 6 | 2 | 7 | 47 | 0.4615 | 0.9592 | 0.7500 | 0.8548 | 1.4207 |
| ≥0.54 | 5 | 2 | 8 | 47 | 0.3846 | 0.9592 | 0.7143 | 0.8387 | 1.3438 |
| ≥0.56 | 4 | 2 | 9 | 47 | 0.3077 | 0.9592 | 0.6667 | 0.8226 | 1.2669 |
| ≥0.57 | 3 | 2 | 10 | 47 | 0.2308 | 0.9592 | 0.6000 | 0.8065 | 1.1900 |
| ≥0.73 | 2 | 2 | 11 | 47 | 0.1538 | 0.9592 | 0.5000 | 0.7903 | 1.1130 |
| ≥0.76 | 2 | 1 | 11 | 48 | 0.1538 | 0.9796 | 0.6667 | 0.8065 | 1.1334 |
| ≥0.78 | 1 | 1 | 12 | 48 | 0.0769 | 0.9796 | 0.5000 | 0.7903 | 1.0565 |
| ≥0.82 | 1 | 0 | 12 | 49 | 0.0769 | 1.0000 | 1.0000 | 0.8065 | 1.0769 |

TP = True positive; FP = False positive; FN = False negative; TN = True negative; TPR = True positive rate (sensitivity); TNR = True negative rate (specificity); PPV = Positive predictive value.

TABLE 7
Fast growth
Common Rates and Indices for each Cut Off Value
Condition Variable Fast Growth at 12 Months
Estimated Prevalence = 0.34

| | Table Counts | | | | | | | | |
|---|---|---|---|---|---|---|---|---|---|
| Cutoff | TPs | FPs | FNs | TNs | TPR | TNR | | | TPR + |
| value | A | B | C | D | (Sens.) | (Spec.) | PPV | Accuracy | TNR |
| ≥0.03 | 21 | 41 | 0 | 0 | 1.0000 | 0.0000 | 0.3387 | 0.3387 | 1.0000 |
| ≥0.07 | 21 | 40 | 0 | 1 | 1.0000 | 0.0244 | 0.3443 | 0.3548 | 1.0244 |
| ≥0.07 | 21 | 39 | 0 | 2 | 1.0000 | 0.0488 | 0.3500 | 0.3710 | 1.0488 |
| ≥0.09 | 21 | 38 | 0 | 3 | 1.0000 | 0.0732 | 0.3559 | 0.3871 | 1.0732 |
| ≥0.09 | 20 | 38 | 1 | 3 | 0.9524 | 0.0732 | 0.3448 | 0.3710 | 1.0256 |
| ≥0.09 | 20 | 37 | 1 | 4 | 0.9524 | 0.0976 | 0.3509 | 0.3871 | 1.0499 |
| ≥0.10 | 20 | 36 | 1 | 5 | 0.9524 | 0.1220 | 0.3571 | 0.4032 | 1.0743 |
| ≥0.10 | 20 | 35 | 1 | 6 | 0.9524 | 0.1463 | 0.3636 | 0.4194 | 1.0987 |
| ≥0.11 | 20 | 34 | 1 | 7 | 0.9524 | 0.1707 | 0.3704 | 0.4355 | 1.1231 |
| ≥0.11 | 20 | 33 | 1 | 8 | 0.9524 | 0.1951 | 0.3774 | 0.4516 | 1.1475 |
| ≥0.12 | 20 | 32 | 1 | 9 | 0.9524 | 0.2195 | 0.3846 | 0.4677 | 1.1719 |
| ≥0.12 | 20 | 31 | 1 | 10 | 0.9524 | 0.2439 | 0.3922 | 0.4839 | 1.1963 |
| ≥0.13 | 19 | 31 | 2 | 10 | 0.9048 | 0.2439 | 0.3800 | 0.4677 | 1.1487 |
| ≥0.15 | 19 | 30 | 2 | 11 | 0.9048 | 0.2683 | 0.3878 | 0.4839 | 1.1731 |
| ≥0.16 | 18 | 30 | 3 | 11 | 0.8571 | 0.2683 | 0.3750 | 0.4677 | 1.1254 |
| ≥0.17 | 18 | 29 | 3 | 12 | 0.8571 | 0.2927 | 0.3830 | 0.4839 | 1.1498 |
| ≥0.18 | 18 | 28 | 3 | 13 | 0.8571 | 0.3171 | 0.3913 | 0.5000 | 1.1742 |
| ≥0.19 | 18 | 27 | 3 | 14 | 0.8571 | 0.3415 | 0.4000 | 0.5161 | 1.1986 |
| ≥0.21 | 18 | 26 | 3 | 15 | 0.8571 | 0.3659 | 0.4091 | 0.5323 | 1.2230 |
| ≥0.22 | 18 | 25 | 3 | 16 | 0.8571 | 0.3902 | 0.4186 | 0.5484 | 1.2474 |
| ≥0.22 | 18 | 24 | 3 | 17 | 0.8571 | 0.4146 | 0.4286 | 0.5645 | 1.2718 |
| ≥0.23 | 18 | 23 | 3 | 18 | 0.8571 | 0.4390 | 0.4390 | 0.5806 | 1.2962 |
| ≥0.23 | 18 | 22 | 3 | 19 | 0.8571 | 0.4634 | 0.4500 | 0.5968 | 1.3206 |
| ≥0.23 | 18 | 21 | 3 | 20 | 0.8571 | 0.4878 | 0.4615 | 0.6129 | 1.3449 |
| ≥0.23 | 18 | 20 | 3 | 21 | 0.8571 | 0.5122 | 0.4737 | 0.6290 | 1.3693 |
| ≥0.25 | 17 | 20 | 4 | 21 | 0.8095 | 0.5122 | 0.4595 | 0.6129 | 1.3217 |
| ≥0.25 | 17 | 19 | 4 | 22 | 0.8095 | 0.5366 | 0.4722 | 0.6290 | 1.3461 |
| ≥0.26 | 17 | 18 | 4 | 23 | 0.8095 | 0.5610 | 0.4857 | 0.6452 | 1.3705 |
| ≥0.26 | 16 | 18 | 5 | 23 | 0.7619 | 0.5610 | 0.4706 | 0.6290 | 1.3229 |
| ≥0.27 | 16 | 17 | 5 | 24 | 0.7619 | 0.5854 | 0.4848 | 0.6452 | 1.3473 |
| ≥0.28 | 16 | 16 | 5 | 25 | 0.7619 | 0.6098 | 0.5000 | 0.6613 | 1.3717 |
| ≥0.30 | 15 | 15 | 6 | 26 | 0.7143 | 0.6341 | 0.5000 | 0.6613 | 1.3484 |
| ≥0.30 | 15 | 14 | 6 | 27 | 0.7143 | 0.6585 | 0.5172 | 0.6774 | 1.3728 |
| ≥0.31 | 15 | 13 | 6 | 28 | 0.7143 | 0.6829 | 0.5357 | 0.6935 | 1.3972 |
| ≥0.33 | 15 | 12 | 6 | 29 | 0.7143 | 0.7073 | 0.5556 | 0.7097 | 1.4216 |
| ≥0.33 | 15 | 11 | 6 | 30 | 0.7143 | 0.7317 | 0.5769 | 0.7258 | 1.4460 |
| ≥0.34 | 15 | 10 | 6 | 31 | 0.7143 | 0.7561 | 0.6000 | 0.7419 | 1.4704 |
| ≥0.34 | 15 | 9 | 6 | 32 | 0.7143 | 0.7805 | 0.6250 | 0.7581 | 1.4948 |
| ≥0.35 | 15 | 8 | 6 | 33 | 0.7143 | 0.8049 | 0.6522 | 0.7742 | 1.5192 |
| ≥0.36 | 14 | 8 | 7 | 33 | 0.6667 | 0.8049 | 0.6364 | 0.7581 | 1.4715 |
| ≥0.39 | 14 | 7 | 7 | 34 | 0.6667 | 0.8293 | 0.6667 | 0.7742 | 1.4959 |
| ≥0.42 | 14 | 6 | 7 | 35 | 0.6667 | 0.8537 | 0.7000 | 0.7903 | 1.5203 |
| ≥0.43 | 13 | 6 | 8 | 35 | 0.6190 | 0.8537 | 0.6842 | 0.7742 | 1.4727 |
| ≥0.43 | 12 | 6 | 9 | 35 | 0.5714 | 0.8537 | 0.6667 | 0.7581 | 1.4251 |
| ≥0.44 | 11 | 6 | 10 | 35 | 0.5238 | 0.8537 | 0.6471 | 0.7419 | 1.3775 |
| ≥0.46 | 11 | 5 | 10 | 36 | 0.5238 | 0.8780 | 0.6875 | 0.7581 | 1.4019 |
| ≥0.51 | 10 | 5 | 11 | 36 | 0.4762 | 0.8780 | 0.6667 | 0.7419 | 1.3542 |
| ≥0.51 | 9 | 5 | 12 | 36 | 0.4286 | 0.8780 | 0.6429 | 0.7258 | 1.3066 |
| ≥0.54 | 8 | 5 | 13 | 36 | 0.3810 | 0.8780 | 0.6154 | 0.7097 | 1.2590 |
| ≥0.55 | 8 | 4 | 13 | 37 | 0.3810 | 0.9024 | 0.6667 | 0.7258 | 1.2834 |
| ≥0.57 | 7 | 4 | 14 | 37 | 0.3333 | 0.9024 | 0.6364 | 0.7097 | 1.2358 |
| ≥0.60 | 7 | 3 | 14 | 38 | 0.3333 | 0.9268 | 0.7000 | 0.7258 | 1.2602 |
| ≥0.61 | 7 | 2 | 14 | 39 | 0.3333 | 0.9512 | 0.7778 | 0.7419 | 1.2846 |
| ≥0.63 | 6 | 2 | 15 | 39 | 0.2857 | 0.9512 | 0.7500 | 0.7258 | 1.2369 |
| ≥0.64 | 5 | 2 | 16 | 39 | 0.2381 | 0.9512 | 0.7143 | 0.7097 | 1.1893 |
| ≥0.65 | 4 | 2 | 17 | 39 | 0.1905 | 0.9512 | 0.6667 | 0.6935 | 1.1417 |
| ≥0.68 | 4 | 1 | 17 | 40 | 0.1905 | 0.9756 | 0.8000 | 0.7097 | 1.1661 |
| ≥0.75 | 4 | 0 | 17 | 41 | 0.1905 | 1.0000 | 1.0000 | 0.7258 | 1.1905 |
| ≥0.79 | 3 | 0 | 18 | 41 | 0.1429 | 1.0000 | 1.0000 | 0.7097 | 1.1429 |
| ≥0.95 | 2 | 0 | 19 | 41 | 0.0952 | 1.0000 | 1.0000 | 0.6935 | 1.0952 |
| ≥0.96 | 1 | 0 | 20 | 41 | 0.0476 | 1.0000 | 1.0000 | 0.6774 | 1.0476 |

Corresponding data for the other proteins of Groups A, B, C and D can be derived in a similar manner.

The logistic regression analysis allowed us to generate indices representing a probability of an individual's AAA being fast growth or slow/no growth in the subsequent 12 months. Setting a cut off value of 0.39 for the probability for slow or no growth (establishing an ‘Aneurysm Slow Growth Index’ or ASGI), the prediction had a sensitivity of 54% (7/13), a specificity 94% (46/49) and an accuracy 85% (53/62). For the prediction of fast growth, the logistic regression analysis generated a probability of an individual's AAA being fast growth in the subsequent 12 months. Using a cut off value of 0.42 for the probability of fast growth (establishing an ‘Aneurysm Fast Growth Index’ or AFGI), the prediction had a sensitivity 66% (14/21), specificity 85% (35/41) and accuracy 79% (49/62).

As taking the diameter of an AAA is routine practice in monitoring an AAA patient, the skilled clinician will, in practice, always have a value for the diameter of a particular patient's AAA available. Accordingly, given an AAA diameter at a particular moment in time, combined with a prediction of growth rate prediction derived from the protein concentrations determined as above, the clinician will be readily able to predict the time period over which an AAA is likely to grow in size to a point at which surgery needs to be considered. The clinician can consequently assess an appropriate time for a follow-up consultation.

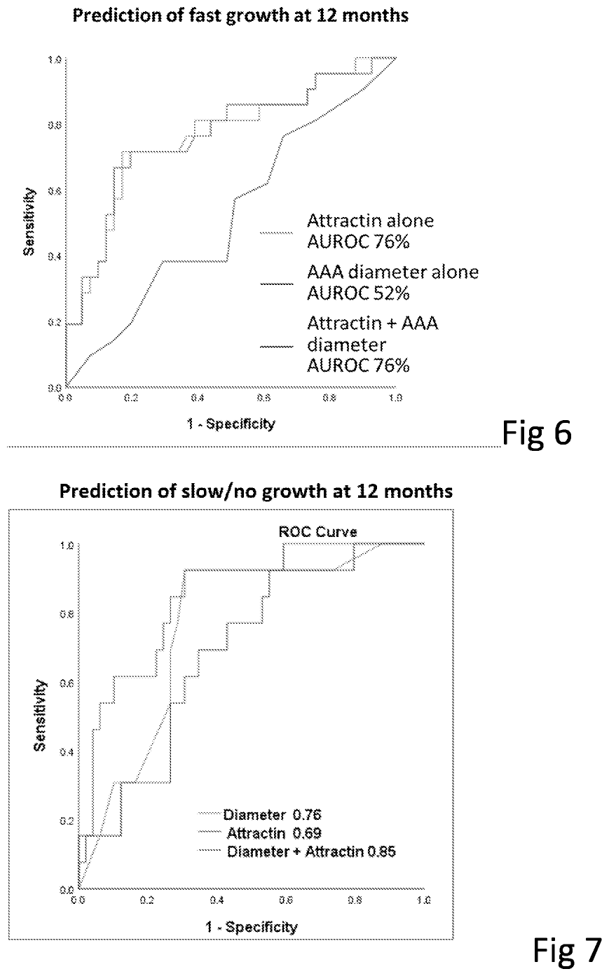

FIG. 6 plots the results obtained using i) attractin level, ii) AAA diameter, and iii) attractin level and AAA diameter in combination, as the input variables. As can be seen, the AUROC for predicting fast growth of AAA at 12 months is 76% based on attractin level alone, 52% based on AAA diameter, and 76% based on attractin level and AAA diameter together. In other words, it can been seen that attractin level alone is an excellent indicator of fast AAA growth. Accordingly, not only can attractin, as our exemplary protein, be highly useful as a predictor of AAA growth when combined with data for the AAA diameter, it is also an excellent indicator of AAA growth alone.

FIG. 7 shows the results using i) attractin level, ii) AAA diameter, and iii) attractin level+AAA diameter in combination, as input variables in the methods of the present invention, showing the AUROC for predicting slow growth of AAA at 12 months is 69% (attractin level alone), 76% (AAA diameter), and 85% (attractin level+AAA diameter).

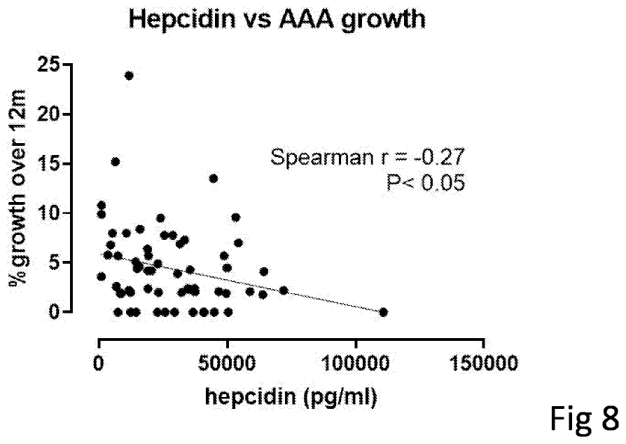

FIG. 8 shows a plot of AAA growth over 12 months against hepcidin level (Spearman r=−0.027, P<0.05), similarly showing a correlation between the growth from the baseline diameter of AAAs and blood hepcidin level at the baseline.

This data supports the use of the proteins in each of groups A, B, C and D circulating in the blood as biomarker indicators of future aneurysm growth. The proteins can be used, either individually or in combinations, to predict future growth rates and, accordingly, provide a physician with information from which they can determine the frequency of follow-up monitoring and timing of surgical procedures to treat the aneurysm.

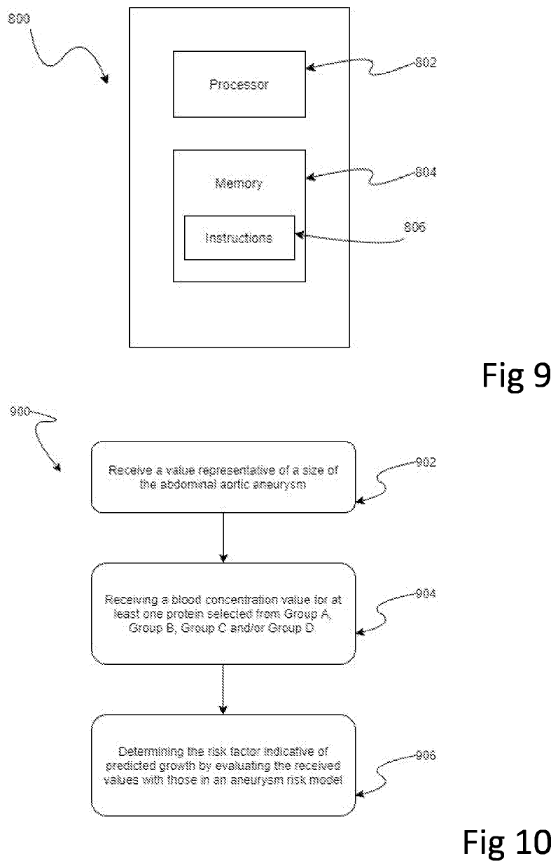

FIG. 9 is a diagram showing an apparatus in accordance with an example of the present disclosure. The apparatus 800 comprises a processor 802 and a memory 804. An instruction region 806 of the memory 804 comprises instructions to cause the processor to carry out steps of the method described herein. The apparatus 800 may further comprise a blood sample analysis module for measuring one or more protein levels in a blood sample or an input module for inputting blood protein level data from an external apparatus. In particular, the apparatus 800 is suitable for use in determining a risk value indicative of predicted growth of an abdominal aortic aneurysm of a patient based on a value representative of or representing a size of the abdominal aortic aneurysm and at least one blood protein value of the patient.

In some examples, the one or more protein levels are determined in respect of at least one protein in at least one of Group A, Group B, Group C and/or Group D.

It will be appreciated that examples described herein can be realised in the form of hardware, or a combination of hardware and software. Any such software may be stored in the form of volatile or non-volatile storage such as, for example, a storage device like a ROM, whether erasable or rewritable or not, or in the form of memory such as, for example, RAM, memory chips, device or integrated circuits or on an optically or magnetically readable medium such as, for example, a CD, DVD, magnetic disk or magnetic tape. It will be appreciated that the storage devices and storage media are examples of non-transitory machine-readable storage that are suitable for storing a program or programs that, when executed, implement examples described herein. Accordingly, examples provide a program comprising code for implementing a system or method as described herein and a machine readable storage storing such a program.

FIG. 10 is a flow diagram illustrating a method in accordance with an example of the present disclosure. The method 900 comprises a first method step 902 of receiving a value representative of or representing a size of the abdominal aortic aneurysm. The method next comprises a second method step 904 of receiving at least one blood protein value of a patient. The method comprises a third method step of determining the risk value indicative of predicted growth by evaluating the received values with those in an aneurysm risk model, the aneurysm risk model relating to a risk value indicative of predicted growth of an abdominal aortic aneurysm for a given value representative of or representing a baseline size of an abdominal aortic aneurysm and at least one given blood protein value. As will be understood the method is performed in the order described herein. In another embodiment the first step 902 and second step may be substituted with one another, or further alternatively performed simultaneously. In a yet further embodiment steps 904 and 906 may be repeated in respect of a plurality of circulating proteins. Such a method may provide a more refined risk value.

### Comparison Between the Above Experiments

The proteins that are significantly different between the experiment and control groups from the above experiment thus fulfil the following criteria:
Group A—are different between those patients with fast vs slow AAA growth.
Group B—the level in systemic circulation is significantly changed after AAA surgery
Group C—present in thrombus
Group D—can be released by the thrombus into circulation

Consequently, we can conclude that these are proteins originating from thrombus of an AAA, and can be used as individual biomarkers within the bloodstream, or in combinations, for the prediction of AAA growth.

The present invention represents a significant breakthrough from previous methods of AAA growth prediction. Prior predictive models, including those set out in our previous application, WO 2017/212210, required the inclusion of a physiological measurement (FMD of brachial artery). Such procedures require a dedicated ultrasound measurement and cannot be derived by plasma sample measurement alone. By focusing on the role of thrombus as a source of systemic mediator release, we have determined novel protein sets that have a specific utility for AAA growth prediction.

Our previous model included nine proteins (Thrombospondin, CXCL10, IL6, IL8, RAGE, MIP1a, MIP1b, leptin, ICAM1) selected by the analysis of plasma samples of fast vs slow/no growth patients using an antibody array (R&D Proteome Profiler). Attractin has the same utility for AAA growth prediction as compared to the other 9 proteins combined. The present invention provides a method requiring, a minimum of only two input variables (AAA diameter and attractin or other marker selected from Group A or other groups or combinations of markers), both of which are readily measured in an outpatient setting. Consequently, with a point-of-care testing device (to measure attractin levels and those of other markers), it is now feasible to apply the present developments at the time of AAA screening and follow-up surveillance scans.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process disclosed.

1. Use of at least one protein selected from at least one of Group A, Group B, Group C and/or Group D as a biomarker for determining a risk value of abdominal aortic aneurysm future growth for a subject; wherein:
Group A is a group of proteins determined to be present at different concentrations in subjects showing fast abdominal aortic aneurysm growth compared with subjects showing slow abdominal aortic aneurysm growth;
Group B is a group of proteins determined to have a concentration significantly lower in the systemic circulation of subjects following abdominal aortic aneurysm surgery;
Group C is a group of proteins determined to be present in thrombus of an abdominal aortic aneurysm; and
Group D is a group of proteins determined to be present in supernatant of an extracted sample of thrombus of an abdominal aortic aneurysm.

2. A method of determining a risk value of future abdominal aortic aneurysm growth for a subject, the method comprising receiving a blood sample of the subject, determining a protein concentration in the blood sample for at least one protein selected from at least one of Group A, Group B, Group C and/or Group D as defined in claim 1; comparing the determined protein concentration with a reference value for the protein and an index of aneurysm growth for the protein; and determining the risk value of future aneurysm growth based on the comparison.

3. An apparatus for determining a risk value of future abdominal aortic aneurysm growth for a subject, the apparatus comprising a data input to receive at least one value of protein concentration of a blood sample of the subject, the protein being at least one protein selected from at least one of Group A, Group B, Group C and/or Group D as defined in claim 1; at least one processor; and a memory comprising instructions executable by the at least one processor to: i) compare the or each protein concentration with a respective protein reference value and an index of abdominal aortic aneurysm growth for the protein; and ii) determining the risk value of future aneurysm growth based on the comparison.

4. An apparatus as claimed in claim 3 further comprising a blood sample analysis module.

5. Use as claimed in claim 1 or a method as claimed in claim 2 or an apparatus as claimed in claim 3 or claim 4 wherein the at least one protein is selected from Group A.

6. Use as claimed in claim 1 or a method as claimed in claim 2 or an apparatus as claimed in claim 3 or claim 4 wherein the at least one protein is at least one protein selected from proteins present in both Group A and Group B.

7. Use as claimed in claim 1 or a method as claimed in claim 2 or an apparatus as claimed in claim 3 or claim 4 wherein the at least one protein is at least one protein selected from proteins present in both Group A and Group B and at least one of Group C and Group D.

8. Use as claimed in claim 1 or a method as claimed in claim 2 or an apparatus as claimed in claim 3 or claim 4 wherein the at least one protein is at least one protein selected from proteins present in Group A, Group B, Group C and Group D.

9. Use as claimed in claim 1 or a method as claimed in claim 2 or an apparatus as claimed in claim 3 or claim 4 wherein the at least one protein is selected from at least two of Group A, Group B, Group C and Group D.

10. Use as claimed in claim 1 or a method as claimed in claim 2 or an apparatus as claimed in claim 3 or claim 4 wherein the at least one protein is selected from at least three of Group A, Group B, Group C and Group D.

11. Use as claimed in claim 1 or a method as claimed in claim 2 or an apparatus as claimed in claim 3 or claim 4 wherein the at least one protein is selected from Group A, Group B, Group C and Group D.

12. Use, method or apparatus as claimed in any one of claims 5 to 7 wherein the at least one protein is selected from Group A and Group B.

13. Use as claimed in any one of claims 1 or 5 to 12 or a method as claimed in any one of claims 2 or 5 to 12 or an apparatus as claimed in any one of claims 3 to 12; wherein Group A comprises the proteins listed in Table 1; and/or Group B comprises the proteins listed in Table 2; and/or Group C comprises the proteins listed in Table 3; and/or Group D comprises the proteins listed in Table 4.

14. Use as claimed in claim 1 or a method as claimed in claim 2 or an apparatus as claimed in claim 3 or claim 4 wherein the at least one protein is at least one protein selected from Table 5.

15. Use as claimed in claim 1 or a method as claimed in claim 2 or an apparatus as claimed in claim 3 or claim 4 wherein the at least one protein is at least one of attractin, Apolipoprotein A4, Complement C8 and HSP90AA5P.
